SEQUENCE LISTING (47 sequences)

SEQ ID NO: 1 - DNA, 20 bp, Artificial Sequence
  Description of Artificial Sequence Synthetic primer
cgcaagaagaaattgccatc

SEQ ID NO: 2 - DNA, 20 bp, Artificial Sequence
  Description of Artificial Sequence Synthetic primer
tccgcgttagaaaaattcca

SEQ ID NO: 3 - DNA, 20 bp, Artificial Sequence
  Description of Artificial Sequence Synthetic primer
ttgtgcagttccgcaaataa

SEQ ID NO: 4 - DNA, 19 bp, Artificial Sequence
  Description of Artificial Sequence Synthetic primer
ttcacctgcaacagcaagc

SEQ ID NO: 5 - DNA, 20 bp, Artificial Sequence
  Description of Artificial Sequence Synthetic primer
ccatgtccgttgaatcacag

SEQ ID NO: 6 - DNA, 20 bp, Artificial Sequence
  Description of Artificial Sequence Synthetic primer
atggcgaaagaagggttaca

SEQ ID NO: 7 - DNA, 20 bp, Artificial Sequence
  Description of Artificial Sequence Synthetic primer
ggcgcttgtaaaattttcgt

SEQ ID NO: 8 - DNA, 21 bp, Artificial Sequence
  Description of Artificial Sequence Synthetic primer
tgcgcaaagttttattgaaca

SEQ ID NO: 9 - DNA, 20 bp, Artificial Sequence
  Description of Artificial Sequence Synthetic primer
ctgtgtgtggcaagcaagtt

SEQ ID NO: 10 - DNA, 20 bp, Artificial Sequence
  Description of Artificial Sequence Synthetic primer
ttgccagcgacaatatttga

SEQ ID NO: 11 - DNA, 20 bp, Artificial Sequence
  Description of Artificial Sequence Synthetic primer
aaagaccgccttaaccacct

SEQ ID NO: 12 - DNA, 23 bp, Artificial Sequence
  Description of Artificial Sequence Synthetic primer
tggcaaggtaaacttctaaagca

SEQ ID NO: 13 - DNA, 20 bp, Artificial Sequence
  Description of Artificial Sequence Synthetic primer
gccactatttctcggacagc

SEQ ID NO: 14 - DNA, 20 bp, Artificial Sequence
  Description of Artificial Sequence Synthetic primer
gtcgtccctttggcaaataa

SEQ ID NO: 15 - DNA, 22 bp, Artificial Sequence
  Description of Artificial Sequence Synthetic primer
aagttttaaccttcgcatttga

SEQ ID NO: 16 - DNA, 21 bp, Artificial Sequence
  Description of Artificial Sequence Synthetic primer
cgcaatgaattccaatttacc

SEQ ID NO: 17 - DNA, 20 bp, Artificial Sequence
  Description of Artificial Sequence Synthetic primer
ttcacgaagagaatcgtcca

SEQ ID NO: 18 - DNA, 20 bp, Artificial Sequence
  Description of Artificial Sequence Synthetic primer
aaagagctgggacttgcgta

SEQ ID NO: 19 - DNA, 20 bp, Artificial Sequence
  Description of Artificial Sequence Synthetic primer
ttgcagttggtggtaacgaa

SEQ ID NO: 20 - DNA, 20 bp, Artificial Sequence
  Description of Artificial Sequence Synthetic primer
cagagttgcatccggaattt

SEQ ID NO: 21 - DNA, 20 bp, Artificial Sequence
  Description of Artificial Sequence Synthetic primer
gaccagtgaaacgtgggaac

SEQ ID NO: 22 - DNA, 20 bp, Artificial Sequence
  Description of Artificial Sequence Synthetic primer
cgtagtgggtatgggcattt

SEQ ID NO: 23 - DNA, 20 bp, Artificial Sequence
  Description of Artificial Sequence Synthetic primer
gaccagtgaaacgtgggaac

SEQ ID NO: 24 - DNA, 20 bp, Artificial Sequence
  Description of Artificial Sequence Synthetic primer
cgtagtgggtatgggcattt

SEQ ID NO: 25 - DNA, 20 bp, Artificial Sequence
  Description of Artificial Sequence Synthetic primer
tctgaaatggccggattcta

SEQ ID NO: 26 - DNA, 21 bp, Artificial Sequence
  Description of Artificial Sequence Synthetic primer
tcgcttttccaattgattttg

SEQ ID NO: 27 - DNA, 20 bp, Artificial Sequence
  Description of Artificial Sequence Synthetic primer
gaatctgcgacttcgattcc

SEQ ID NO: 28 - DNA, 20 bp, Artificial Sequence
  Description of Artificial Sequence Synthetic primer
gcaaatgaacccagctatgg

SEQ ID NO: 29 - DNA, 20 bp, Artificial Sequence
  Description of Artificial Sequence Synthetic primer
aattctccgctgggattttt

SEQ ID NO: 30 - DNA, 20 bp, Artificial Sequence
  Description of Artificial Sequence Synthetic primer
ttggttctggagttccaagg

SEQ ID NO: 31 - DNA, 20 bp, Artificial Sequence
  Description of Artificial Sequence Synthetic primer
gaccagtgaaacgtgggaac

SEQ ID NO: 32 - DNA, 20 bp, Artificial Sequence
  Description of Artificial Sequence Synthetic primer
cgtagtgggtatgggcattt

SEQ ID NO: 33 - DNA, 22 bp, Artificial Sequence
  Description of Artificial Sequence Synthetic primer
caggatatattttgagcgagca

SEQ ID NO: 34 - DNA, 22 bp, Artificial Sequence
  Description of Artificial Sequence Synthetic primer
ttttcgatttcttgtttttcca

SEQ ID NO: 35 - DNA, 20 bp, Artificial Sequence
  Description of Artificial Sequence Synthetic primer
cgcctttcgttcatgacaat

SEQ ID NO: 36 - DNA, 20 bp, Artificial Sequence
  Description of Artificial Sequence Synthetic primer
cgaagaacttgccagtgtga

SEQ ID NO: 37 - DNA, 660 bp, Leptospira interrogans
ataaaactttaaaaggaaagtttttacgaaatttatttaaaggattctatattcttctacaatttcacgaagagaatcgtccatctcacgaataaaacgaagttcttgagtcattctccacattgtaattgaaccgtggtatgccattaaaattcttctcgccacgtattggatgtccatatttcttttgagttgtcccgatccgatcgccttttgcagataatcgctgatcatacgaatccatttgtttacgatgtcttttaaaaattcagtatattcaggatcggagtccattacttgacttgcaaaaccagcaaaaggacaacctacaaattctccgtgacggatttgtttttctttgagaatcacccaggctcttaaaaattctcctaggtctggataacgatccatcaaactttgaagatcgctaagagttttttcttcctgacgagctagatacgcaagtcccagctcttttttagaagggtagtgatcgtagagacttgcagcaacagaattggattcctgaatgatttgcctcatacctgtactggaaaaaccttgtttgtaaaaaagagaagtagcggtatctaaaattctttcccgaactccagttccggattctttttttttacgcggttgagttttcatagaggaacg

SEQ ID NO: 38 - DNA, 1380 bp, Leptospira interrogans
ccaatcaaagcgtaagttcgtcttgttgaacttaacaaacgcaaattgatcctatgtccggaaatgataaaaagattctagaacaaaattctcacggggaatttgaactaacagcttacggagaattcttaagttatttccatactcatattcaactttttaacggtcttatctctggtaagaaaatttctcccacagaccaggaagttcttaaacaaaaagtacgttcttatattgtaagtaatatccaaaagacggaacaattttttgatcacctgcctaagttcgcagaatttctaggaatgtcccaatctgatctttccgcgtttatgacgaaaaacttcatgaacacgcacgctgggatcaaaaacaaactcatcgaacaggaaaaggcaaacataggtaaacctaaaaagaaaagatattcccgcatttcggaagaaattttggaaacgatcggagaattggttcctccaggtaaaaggtttatcggaatggaaggttatgtagtccttagggacgatgcgactggaaaagatcttgaaccttctatttcttctttcggcgaaacacctaaacaaacgaacgaggaaaatacgccgggtgtcgttaaaaaaacagcaccacaagcttctacccccgccgtgcctttgaaaaagcagactgagaaattgattcttactgagatcttggaaaaatttggttctgaattttccggaaaggttttagaattaaaaaaggaagagtttgaggaagcggaaataaccaatcaaattgcagttggtggtaacgaagaattgttaaccgaagtagaggatcttcaatttgaaggtttcgaagagcctacttttttagaagatacttcgtttgaaccggaagagccacctgtgaatattccgttttctaaatatatggaaagtataaaccgtgttcgacagtttcaaaaagacggtcaaccggacgcatataaaaaatgggtgatggctcttcctccggaactcagtgcacttgttcaacttcattcttttgttttaaaggaaatgaaaaacgaacctttggattggaattctaccctttcctcgatttcttcccgaatgggattaacggagaaaagactttggaaggttttagatttgacccgtatttttgccgaactcagggcaggtttggagaaggcttttgtatcttctaaaactgctggtccaggaatggaagaacttgtaaaaaaagcctggcctcatattctcaaaatcttcgaagaatacccaaatacttcttctttgcaccaaaagttagatcaactttttagtaaaattccggatgcaactctgagaaaaaaactttccgatctatttcttcctcttcttcaaaaactctgat

SEQ ID NO: 39 - DNA, 600 bp, Leptospira interrogans
atgagttttaaatttgatgatagccgcctaatagtgattttatgtagaaaaccactctaagtctaatgcgtaaaaataggattctggttcacagtgtagttttcgagtgacttggttgtttgacagatctaggtgcaggatataactgtaggaccagtgaaacgtgggaactcccgcccgaaccgtattcaagatagaattaccgactccgagggcttccaactttaaaagataattaaaaagataacattttgatacaccgtgagcctgagccaatgttcccaaaagagtccaacttgcagaaggaacacgaacgctgattcgtttcatttttaatttgcctggactgggttgatacaaagttctgtccgcttttttaccgagacgttccgacgaagaaagatatttcgcataaactttcaaaagtgtagggatccttttcgtaagtagcctcacgtctttttcagaaaaaagaagccaagtatcttccggaattaaaagagtcaccgtttcagaacaattcttttggaatgtagaactaatttcatgatcagagttgagtaataaaatgcccatacccactacggttctcgacaagag

SEQ ID NO: 40 - DNA, 600 bp, Leptospira interrogans
ttttatgtagaaaaccactctaagtctaatgcgtaaaaataggattctggttcacagtgtagttttcgagtgacttggttgtttgacagatctaggtgcaggatataactgtaggaccagtgaaacgtgggaactcccgcccgaaccgtattcaagatagaattaccgactccgagggcttccaactttaaaagataattaaaaagataacatttcgatacaccgtgagcctgagccaatgttcccaaaagagtccaacttgcagaaggaacacgaacgctgattcgtttcatttttaatttacccggactatgttgatacaaagttctgtccgcttttttaccgagacgtttcgacgaagaaagatatttcgcataaaccttcaaaagtgtagggatccttttcgtaagtagcctcacgtctttttcagaaaaaagaagccaagtatcttccggaattaaaagagtcaccgtttcagaacgattcttttggaatgtagaactaatttcatgatcagagttgagtaataaaatgcccatacccactacggttctcgacaagagtaagacggcttaaatttgaatcaaaaaaatcactcat

SEQ ID NO: 41 - DNA, 660 bp, Leptospira interrogans
atttgaaacctaaaatagcaattcaaatctacaaaagtagactcattttatcaaaagaagaccgtggttttcaaaatagatccgttctgaaatggccggattctattgaatcttctacatctaaaatagaagacgattgtgtccgctttatttccaatctagattttagtacaacagaatcgtttgcaaattcagaatttttcgttactttagattatcatccagagactaaaattcccaaaaaccagttctatgacgatcttgacgaagtttcatttaacttttcaaaattaactagattcaaatttgatatcttcgaatttgaaaaaatgagttcggatcaagtaaatttattttttaatggaacaaatttgggtattcctaaaagacaatcttttcaaatagccagcctaagtctaaattcaccggttagaatcaaaatcaattggaaaagcgacttttctttttctcagggaaaagaaagacaattttacgaactagattatatcgttgaatttaaaggtatatttgaaacttttttgagagaaaatcccaaaacagaaacctttctcaaatatatccaaatatcgaactgtaaagaaatagatcttagaaaaattctcaattaatacgtgtttgatgcaaaaaaattagattttt

SEQ ID NO: 42 - DNA, 660 bp, Leptospira interrogans
tttaattgatttcatccctataatccataaccaaatacattagagtttcttgatctgttggattggaatattcatgaggaatatcggctctgaaaaaaacggaatctctaggttccaactctacaactttttcaccaaccctaagacgaagtttaccggaaacaacaacaatgttttcagtagtgcctgatgagtgggaatctgcgacttcgattcctcccggttttaaaatgagttcataaaactctgtttttcgatttccgttataaggaaaaagagctcggctggaaaaaactttagagctagaaaacaaaaccttagtgttttccgccttcataacaattacaccttcggtacctttttctttgagtaattcgctaaaaggcacattgagtccattcgcaattttccaaagaacggaaatggtcggaacacttttcccttgttcaatttgagaaagcatcgccctgctgactccacatctagaagccagtttgtccaaagagaaccctttagtatggcgaatgagttttaaattttctttgacgacttcagtaatatgttcggaggaagactctttcccctcttctaattcttccatagctgggttcatttgctcttttttatctaatatagcagaaatttgtcaatccaaaacta

SEQ ID NO: 43 - DNA, 1260 bp, Leptospira interrogans
caagttatcttaggtcaaaagaattgttagaggtgagagtagaatggatttttttccggatctgaattttcttgattatttaatttttgtaaaagaagtattaaaccggtatctagaatatctttactctgcaagtatagtaattagttttttaaccggtctttctggtttttacaaagcaaccaaaaatcgtttaaactttgttcgaggaatttttttaatgagcgcttgttttctaattatcggacacagtaaagctttttcattgatgacttcggatccaaatggaattttaagtctcgaaaagcgtgtctttttttcctattactacggatttgcagtttgtgcctctttatcttccaccttctacgtcatgtatctttttggtgctttgaaaaatcctctacgtttgagcctatatagttttctttcttttccaacaatctttcttatttctttaatgatagaaaacttaatttcaatcgtttactttggaaagtcagttctttttaccttacaattctccgctgggatttttaatctttattatgttatcaaatataagatgaaaaaaatctatctcaattatccaattcaaaattttacactttgtacagcgcttatcgtacattcgatcggaatgtggttaggcactccaaacttgatgattcttgcactttcttttacaggatttttcattatttattttttcatattggaatacaatcatcctgaattttggaaagtgagtttttccagcgaactgatagaaattaaatcagaagataataaaattcaaactctacaaaccaatactagttctaaaaatttagtagaacgtttagacatatctcgtatcgaagaaaaaattcaaaaatttgtggaagatagagaatatttagacgaagaaattcgcttgtccgatttttcagcttatatcggactttcacttcaccaagcttcttattatttgaacaattataaggatctcagctttacagatttcttaagcttccatagactagaagaagcaaaacgtatgattgaacaaagaccagatattaatcttttagaagtggctttagcaagcggtttcaattctccttcttcttttagaagagcttgtctaaaatttaccggaaaacctccaaaagaatttagaaactacgtacttcatcaaacaactccttctattaccttggaactccagaaccaattaggataattttttcgttccatctaatttgattgatagaacga

SEQ ID NO: 44 - DNA, 660 bp, Leptospira interrogans
aatctaggaacttctacgaaatgagttttaattttgatgataaccgcctaatagtgattttatgtagaaaaccactctaagtctaatgcgtaaaaataggattctggttcacaatgtagttttcgagtgacttggttgtttgacagatctatgtccaggatataactgtaagaccagtgaaacgtgggaactcccgcccgaaccgtattcaagatagaattaccgactccgagggcttccaactttaaaagataattaaaaagataacatttcgatacaccgtgagcctgagccaatgttcccaaaagagtccaacttgcagaaggaacacgaacgctgattcgtttcatttttaatttacctggactgggttgatacaaagttctgtccgcttttttaccgagacgttccgacgaagaaagatatttcgcataaactttcaaaagtgtagggatccttttcgtaagtagcctcacgtctttttcagaaaaaagaagccaagtatctttcggaattaaaagagtcaccgtttcagaacgatttttttgaagtgcggaactaatttcatgatcagaattgagtaataaaatgcccatacccactacggttctcgacaagagtaagacggctcaaatttgaatcaaaaaaatcactcattca

SEQ ID NO: 45 - DNA, 480 bp, Leptospira interrogans
gattagtattattttaatactaatgtcaattaaaaagtattattccaggatatattttgagcgagcaaaaaaaaagactaaaaaccatacttttagatttcaaaggaaatcaaagggagtttggagtcacaattggcaaatccaaacagacaatcagtggctggctaagtggtagatttccaataccagaggatgctgcaattacaatagaaatggttcatggataccgaagacaatggcttctcgaaggagaacttccagaaaaagtaactagacgaatacaaacatctcgaacaagaacaaaagaatttgaactagaaaagactttgttaaaaaaaataacttctaaggagggccttcctaaaatgattgaaatacttacagtacttccaaaaaaagaatttgaaatcgcccaaagatttatttttagcctggaaaaacaagaaatcgaaaacaattagaatatatcctatcaagaat

SEQ ID NO: 46 - DNA, 1020 bp, Leptospira interrogans
aaaacattggcacgatcgtatctaatgaagaatcgattctaattctctcagaatccgctttgccaaatgagtaggacgttccaaaattacattttttccaaatggaagaattgtttccaaaaaccagttttcttcccgaattttggctttggataaatgataaacgacattatccaatttttgagttttttcggttctttccaaatcaagtttacgattgagattataaaacacttcccgagtatgccaaatttctgcgattccggatgaattctcccaacttttacgaaattcttcaaattctcgaatataatctgattttttttgaccagctggttgcaagatcgggtccgaaccaatttttaaggaaagaatattgtccaagcgaaaatttcgaggcgcctttcgttcatgacaatatcctaataaataatcttctaatgaatgaaataaaaaccaaggatctacttttctaagagtagatttttcgcccattctggattgatattccaggattaaagatttttcgttttgaatcgcattttgaatattagttttgtaaaccgaaagtgcttcctggccagcaatgggaacaatagaaatgattttctgaagtattttacgagcagttgaatttgtttttggatcggagctggcttctacaacttcttctaaaatttttctaagtgttagccattctcgaatactcaaacgtaaactagaatcaaaacgaaaaggtaaaatcaaaccaaatgtatccgtttctgaatcataatcgacttggatcaaatcggcgacgtgaggagttgtccccagaaaaaaaagttctcccaattgttcttttaaatctttttgattgttgattccggtcacactggcaagttcttcgagagtcattcgtttaccatctcgaagatggcgaatcagattcaatttaaaattcagtcgaactgtacttggattcacacctcaaatgtaaaaatggcacacattctgtaaaccggaaatgtaagaatcttg

SEQ ID NO: 47 - DNA, 600 bp, Leptospira interrogans
attttaggctctattcaacaattgtatgaataaaattttctttagaaggtaaaaatgaaattttggtttccacaagaaagacgattttattttttttcgatgtatatctttttaattttactttggatcattgaagaagttttaaccttcgcatttgatattaattggatagaaagatctcaggcttattttactacgatcgaagcagcattcggtctgttatctattataggaatttattttttatttcaagaaattagaaacacaaaaacggatattgaatctgctaaaataatgatcgaaggactcaaaaataaaaatcaatttttagttcaaaccaatcaatctttctgggaatctcttcaaagacaattggaagaatgggacctctccgataaagaaaaagaaattgcacttttacttctaaggggtatgtctaatcaccaaattgctgctattcgaggaaaaagtttaaaaacaatcgagaaccaaacattttctatctatcaaaaatctggaactactggtaaattggaattcattgcgtatttcatttctcctttactacccgaagaagattgatctataatctttctcgta

(19) United States
(12) Patent Application Publication
(10) Pub. No.: US 2006/0275795 A1
(43) Pub. Date: Dec. 7, 2006

(54) Determination and potential control of pathogenic bacteria or bacterial strains

(75) Inventors: Dongyou Liu, Starkville, MS (US); Mark L. Lawrence, Starkville, MS (US); Frank W. Austin, Starkville, MS (US); A. Jerald Ainsworth, Starkville, MS (US); Lanny W. Pace, Brandon, MS (US)

Correspondence Address: DLA PIPER US LLP;ATTN: PATENT GROUP, 1200 NINETEENTH STREET, NW, WASHINGTON, DC, 20036, US

(73) Assignee: Mississippi State University

(21) Appl. No.: 11/372,187

(22) Filed: Mar. 10, 2006

Related U.S. Application Data
(60) Provisional application No. 60/660,332, filed on Mar. 11, 2005.

(51) Int. Cl. C12Q 1/68; C12P 19/34

(52) U.S. Cl. 435/6; 435/91.2

(57) ABSTRACT

The present invention is directed to methods to detect and/or determine bacterial pathogenicity and species identity based on genes that encode transcriptional regulators or putative transcriptional regulators.

This application claims priority from U.S. Provisional Application Ser. No. 60/660,332 filed Mar. 11, 2005. The entirety of that provisional application is incorporated herein by reference.

This invention was made with Government support under 58-6202-5-083 awarded by the U.S. Department of Agriculture. The Government may have certain rights in this invention.

### FIELD

This invention relates to the fields of Microbiology and Moleculary biology. Particularly, the present invention relates to methods of detecting bacterial pathogenicity and species identity based on genes that encode transcriptional regulators.

### BACKGROUND

There is a need for the detection of bacteria pathogens that can cause diseases in mammals and plants. However, traditional methods to detect the presence of bacterial pathogens require an extended period of time for growing these bacteria from a background of competing microorganisms and an increase in bacterial cell numbers to more readily aid in identification.

For example, the standard FDA procedure for detection of Listeria in food products takes 4 days and the identification of Listeria colonies is done by eye (Bacteriological Analytical Manual, 7th Ed., 1992; Chapter 10). Other bacteria, such as Leptospires, take eight weeks or longer to grow in laboratory and have stringent nutritional requirements.

An alternative detection method is to use immunology-based assays, such as ELISA. The immunology-based procedures reduce or eliminate the requirement of a growth period, but are not very efficient in detecting low numbers of bacteria. Moreover, ELISA often relies on the use of bacteria of various serogroups as antigens, and therefore any serogroups that are not included in the antigen preparations may give false negative results.

Accordingly, there is an urgency to develop and apply screening tests with enhanced sensitivity and specificity for pathogenic bacteria in an effort to promptly contain and eliminate bacteria infections.

The development and application of molecular methods involving nucleic acid amplification (such as PCR) have enabled vast improvement in the laboratory detection and identification of bacteria. The deciphering of complete genomes of various bacteria pathogens also opens new avenues for improving the detection and potential control of these pathogenic bacteria.

### SUMMARY

The foregoing needs are met to a great extent by methods for detecting a pathogenic species of bacteria or pathogenic strains of a species of bacteria by polymerase chain reaction (PCR) using primers specific for the DNA sequence of genes that encode putative transcriptional regulators.

In one embodiment, a method for detecting the presence of Listeria monocytogenes in a sample comprises the steps of subjecting the sample to PCR amplification using primers designed to target a putative transcriptional regulator gene Imo0733; and detecting the presence of an amplification product of the Imo0733 gene as an indication of the presence of Listeria monocytogenes.

In other embodiments, methods for detecting the presence of Pasteurella multocida, Staphylococcus aureus, Streptococcus pyogenes, Enterococcus faecalis, or pathogenic Leptospira strains in a sample by subjecting the sample comprise the steps of PCR amplification using primers designed to target a putative transcriptional regulator gene and detecting the presence of an amplification product of the putative transcriptional regulator gene as an indication of the presence of Pasteurella multocida, Staphylococcus aureus, Streptococcus pyogenes, Enterococcus faecalis, or pathogenic Leptospira strains.

### BRIEF DESCRIPTION OF THE FIGURES

FIG. 1. Examination of PCR products generated using L. monocytogenes specific primers Imo0733F and Imo0733R.

FIG. 2. Determination of the detection limit for primers Imo0733F and Imo0733R using L. monocytogenes genomic DNA as template.

FIG. 3. Agarose gel electrophresis of DNA products generated in PCR with P. multocida specific, putative transcriptional regulator gene primers Pm0762F and Pm0762R.

FIG. 4. Agarose gel electrophoresis of DNA products amplified in PCR using primers from Staphylococcus aureus putative transcriptional regulator gene sa0836.

FIG. 5. Electrophoretic examination of S. pyogenes-specific PCR products generated with primers Spy1258F/R.

FIG. 6. Agarose gel electrophoretic of amplified DNA products by using E. faecalis specific primers Ef027F/R in PCR.

### DETAILED DESCRIPTION

The practice of the embodiments described in further detail below will employ, unless other wise indicated, conventional methods of microbiology, molecular biology, and immunology within the skill of the art. Such techniques are explained fully in the literature. All publications, patents and patent applications cited herein, whether supra or infra, are hereby incorporated by reference in their entirety.

One aspect relates to the use of transcriptional regulator genes for specific detection and potential control of pathogenic bacteria including Listeria monocytogenes, Pasteurella multocida, Staphylococcus aureus, Streptococcus pyogenes, Enterococcus faecalis, and Leptospira.

Listeria monocytogenes is an opportunistic bacterial pathogen responsible for causing a significant proportion of human foodborne diseases worldwide. Pregnant women, neonates, immuno-suppressed individuals and the elderly are particularly prone to L. monocytogenes infections. The fact that it is found in a variety of food sources, such as vegetables, milk, cheeses, fish, meat and poultry products; and are can tolerate high concentrations of salt, extreme pH and temperature; are of particular concern to the food industry and public health regulatory agencies (Doyle, et al., J. Food Prot., 64:410-429 (2001)). Recent outbreaks of listeriosis due to contaminated foods have highlighted the importance of continuing surveillance of opportunistic pathogens such as L. monocytogenes in ready-to-eat food products (Robinson, et al., Encyclopedia of Food Microbiology (2000); Vasquez-Boland, et al., Clin. Microbiol. Rev., 14:584-640 (2001)).

Pasteurella multocida is a Gram-negative, nonmotile, facultatively anaerobic coccobacillus that forms part of the commensal flora in the oral cavity, upper respiratory and gastrointestinal tract of domesticated and wild animals. It is capable of producing septicemic or respiratory diseases in animals by infecting organs and tissues that have been previously weakened by stress, injuries or other microbial infections (Rimler, et al., Pasteurella and Pasteurellosis, (1989); Bisgaard, Zentbl. Bakteriol., 279 (1993)). This species is also an opportunistic pathogen to humans; bite and scratch wounds from pet animals such as cats and dogs can result in severe localized infections (Rimler, et al., Pasteurella and Pasteurellosis (1989); Frederiksen, Zentbl. Bakteriol., (1993)).

Staphylococcus aureus is a common, Gram-positive species that is pathogenic to both man and animals. Through generation of enterotoxins and superantigens, it can provoke severe immune responses in the host, resulting in some unique and occasionally fatal disease entities such as toxic-shock syndrome and staphylococcal scarlet fever (Robinson, et al, Encyclopedia of Food Microbiology, (2000)). Apart from being a leading source of gastroenteritis via contaminated foods (Le Loir, et al., Genet. Mol. Res., 2:63-76, (2003)), S. aureus has also been responsible for an increasing number of hospital-acquired infections due to its ability to acquire and develop resistance to antibiotics (Robinson, et al, Encyclopedia of Food Microbiology, (2000); Montesinos, et al., Infect. Control Hosp. Epidemiol., (2003); Strommenger, et al, J. Clin. Microbiol., 41:4089-4094 (2003)). In particular, the emergence of methicillin-resistant S. aureus (MRSA), first noted over two decades ago, has made it one of the most important human bacterial pathogens of modern times.

Streptococcus pyogenes (Group A streptococcus or GAS) is a Gram-positive, opportunistic bacterial pathogen that is transmitted via direct contact or respiratory droplets. Although it may exist in the respiratory tracts and skin of humans without causing obvious illness, this bacterium will rapidly multiply and spread in the host tissues in cases where host defenses become weak or defective, producing a variety of clinical diseases (Cunningham, Clin. Microbiol. Rev., 13:470-511 (2000); Schwartz, et al., Lancet, 336:1167-1171 (1990); Musser, et al., Emerging infections, pp. 185-218 (1998); Bemer, et al., Eur. J. Pediatr., 158:527-529 (2000)). In its acute form, S. pyogenes infection often appears as pharyngitis, scarlet fever, impetigo or cellulitis. Any delay in treating the acute S. pyogenes infection may result in a more systematic, invasive, toxigenic form of disease, with manifestations ranging from bacteremia to streptococcal toxic shock syndrome. In a rare, worst case of scenario, acute rheumatic fever or acute giomerulonephritis may develop as immune-mediated post-streptococcal sequelae (Cunningham, Clin. Microbiol. Rev., 13:470-511 (2000)). As invasive GAS infections have emerged as an increasingly important health concern worldwide, it is vital that improved diagnostic techniques are available for early diagnosis and prompt treatment of infections due to S. pyogenes.

The genus Enterococcus comprises a large number of Gram-positive bacterial species that are present in the gastrointestinal tract in humans and animals as normal flora, and also form an important part of the lactic acid bacteria in foods (Murray, Clin. Microbiol. Rev., 3:46-65 (1990); Jett, et al., Clin. Microbiol. Rev., 7:462-478 (1994); Franz, et al., Int. J. Food Microbiol., 88:105-122 (2003)). With their extraordinary ability to obtain genetic elements encoding virulence traits or antibiotic resistance from each other and also from other bacteria, enterococci, especially E. faecalis and E. faecium, have emerged as significant human pathogens in many parts of the world, causing bacteremia, endocarditis and other nosocomical infections. Of particular notice, E. faecalis alone accounts for 80-90% and E. faecium for 10- 15% of human enterococcal infections, with E. gallinarum, and E. casseliflavus being other clinically relevant enterococcal species (Murray, Clin. Microbiol. Rev., 3:46-65 (1990)).

The genus Leptospira represents a diverse group of spirochete bacteria with varying pathogenic potential. Being ubiquitous in the environment, Leptospira is found in a wide range of feral and domestic animals, which act as reservoirs for this zoonotic pathogen. Leptospiral infection in humans invariably results from direct or indirect contact with the urine of infected animals. Although human leptospirosis often presents as flu-like episodes with sudden onset of fever, headache and chills, failure to promptly undertake antibiotic treatment for the infection may lead to severe, sometimes deadly, renal, hepatic and pulmonary damage in patients (Levett, Clin. Microbiol. Rev., 14:296-326 (2001)). With its non-specific symptoms, leptospirosis has been largely unrecognized and neglected for a considerable length of time. However, following development of improved detection methodologies in recent decades, the presence of leptospirosis in man and animals is better documented. In fact, leptospirosis is now considered as an emerging infectious disease worldwide.

A method for detecting pathogenic bacteria such as with PCR amplification uses primers designed to target transcription regulators of the pathogenic bacteria. Transcriptional regulators are specialized DNA binding proteins that play an essential role in directing gene expression within bacteria for their adaptation and survival in different environmental conditions. Because different bacterial species and subspecies are able to adapt to different and sometimes highly specialized environmental niches, unique transcriptional regulators would be required for each group of bacteria. Therefore, it is likely that transcriptional regulators may be genus-, species-, or subspecies- specific, with potential for diagnostic applications.

In an embodiment, the method comprises the steps of identifying a transcriptional regulator or a putative transcription regulator gene in a bacteria strain of interest, subjecting a sample to PCR amplification using primers designed to target the transcriptional regulator gene, and detecting the presence of an amplification product of the transcriptional regulator gene as an indication of the presence of the bacteria strain of interest.

The transcription regulator or a putative transcription regulator can be identified by conducting a Blast search on the genomic sequence of the bacteria strain of the interest and selecting those transcriptional regulator genes that display no homology with other DNA sequences at GenBank. Oligonucleotide primers can be designed from the selected genes using commercially available software, such as the Primer3 software (Whitehead Institute for Medical Research, Cambridge, Mass.). The PCR amplification conditions can be optimized based on the specific primer sequences.

As is well-known to one skilled in the art, the sample can be a bacterial culture sample, a tissue sample, a body fluid sample, a food sample, or a field sample. In one embodiment, DNA is extracted from the sample and is then subjected to PCR amplification. In another embodiment, the sample is subjected to direct PCR amplification.

Since transcriptional regulators and other regulatory proteins are essential components in the regulation of RNA synthesis and gene expression within bacteria, they may be potentially useful targets for treatment and control purposes. Therefore, it is also within the scope of this invention to use virulence-specific Leptospira genes or their derivatives in the inhibition of growth, reduction of pathogenicity, treatment, and prevention of leptospirosis caused by pathogenic Leptospira species.

For example, one possible treatment strategy would involve using pharmaceutically active agent(s) that would inactivate or alter the function of one or more of the proteins encoded by the above listed genes, which would either kill the pathogenic Leptospira or render it susceptible to the host immune system. One possible vaccine strategy would involve altering one or more of the above listed genes or promoter(s) for one or more of the above listed genes such that expression of the encoded protein(s) would be completely disrupted or altered. The alteration or disruption of expression would render pathogenic Leptospira avirulent and effective as a live attenuated vaccine.

These strategies may be suitable for the control of any bacterial pathogen that has identifiable genes encoding transcriptional regulators which are specific to said bacterial pathogen or pathogenic strains of a bacterial species. Examples of such bacteria include, but are not limited to Listeria monocytogenes, Pasteurella multocida, Staphylococcus aureus, Streptococcus pyogenes, and Enterococcus faecalis.

The commercial value of these methods lies in their applicability for rapid and specific laboratory detection and diagnosis of a broad spectrum of pathogenic bacteria. This overcomes the deficiency in the prior art by broad specificity for all instead of a limited few pathogenic species.

The following publications are incorporated herein by reference:

Liu, D., et al., Journal of Medical Microbiology 52:1065-1070 (2003); Liu, D., et al., International Journal of Food Microbiology 9: 297-304 (2004); Liu, D., et al., Journal of Microbiological Methods 58, 263-267 (2004); Liu, D., Lawrence, M. L., et al., Letters in Applied Microbiology 40: 69-73 (2005); Liu, D., et al., Research in Microbiology 156:564-567(2005); Liu, D., et al., 156: 944-948 (2005) and Liu, D., et al., Canadian Journal of Microbiology 54, in press (2006).

The present invention is further illustrated by the following examples which should not be construed as limiting. The contents of all references, patents and published patent applications cited throughout this application, as well as the Figures and Tables are incorporated herein by reference.

### EXAMPLE 1

### Use of PCR Primers Derived from a Putative Transcriptional Regulator Gene for Species-Specific Determination of Listeria monocytozenes

Bacteria.

Listeria and other bacterial reference strains were obtained from the American Type Culture Collection (ATCC) or the National Collection of Type Culture (NCTC). Some environmental isolates were obtained by us (Erdenlig, et al., Appl. Environ. Microbiol., 65:2827-2832 (1999); Erdenlig, et al., J. Food Prot., 63:613-619 (2000)), and other food and clinical isolates were provided by Dr. Catherine Donnelly (Department of Nutrition and Food Sciences, University of Connecticut) and Dr. Robert Mandrell (United States Department of Agriculture-Agricultural Research Service, Albany, Calif.). A total of 52 Listeria strains were examined (Table 1). These included 30 L. monocytogenes, ten L. innocua, three L. grayi, two L. ivanovii, three L. seeligeri, and five L. welshimeri strains. Moreover, 15 other common gram-positive and -negative bacterial species were analyzed to verify the specificity of the PCR primers developed.

Extraction of Genomic DNA.

Genomic DNA was isolated from stationary phase cultures by phenol/chloroform extraction and isopropanol precipitation. L. monocytogenes or other bacterial species were grown on 5% sheep blood agar plates (TSA II, Becton Dickinson Microbiology Systems, Cockeysville, Md.), and several colonies were used to inoculate 25 ml of brain heart infusion (BHI) broth (Difco Laboratories, Detroit, Mich.). Cultures were incubated at 37° C. overnight with shaking, and bacteria were pelleted from the entire 25 ml and resuspended in 2.5 ml of 1×TE (10 mM Tris-HCl pH 8.0 and 1 mM EDTA pH 8.0) containing 2 mg/ml lysozyme (Sigma, St Louis, Mo.). Each tube was incubated at 37° C. for 30 min, and 250 μl of 10% SDS and 25 μl of 10 mg/ml proteinase K (Sigma) were added. After incubation at 56° C. for 2 hours, one volume of phenol/chloroform/isoamyl alcohol (25:24: 1) (Sigma) was added to each tube. Following centrifugation, the supernatant was transferred to a fresh tube, and one volume of isopropyl alcohol and 150 μl of 5 M NaCl were added. DNA was pelleted by centrifugation and washed with 3 ml of 70% ethanol. The purified DNA was resuspended in 1×TE, and DNA concentrations were determined spectrophotometrically at absorbances of 260 and 280 nm in a GeneSpec I (Hitachi Genetic Systems, Japan). Two micrograms of purified DNA from each bacterial strain was diluted in distilled water to 10 ng/μl for PCR analysis.

Identification of L. monocytogenes Specific Genes.

With the complete genomes of L. innocua strain CLIP (serovar 6a) and L. inonocytogenes EGD-e (serovar 1/2a) being available at GenBank (GenBank/EMBL accession numbers AL592022 and AL591824) (Glaser, et al. Science, 249:849-852 (2001)), a comparative genomic analysis was conducted using annotation data and BLAST searches to select gene(s) that would be unique to L. monocytogenes. Specific primers from the selected genes were designed with Primer3 software (Whitehead Institute for Medical Research, Cambridge, Mass.), and synthesized by Sigma Genosys (The Woodlands, Tex.).

PCR Amplification.

PCR amplification was performed in a 25 μl volume using a GeneAmp PCR System 2400 (Perkin Elmers, Foster City, Calif.). Each reaction mixture consisted of 0.5 U Taq DNA polymerase (Fisher Scientific, Houston, Tex.), 1× PCR buffer (containing 10 mM Tris-HCl pH 9.0, 50 mM KCl and 1.5 mM MgCl₂), 50 μM dNTPs, 25 pmol each primer, and 15 ng (1.5 μl) of template DNA. Reaction mixture with no template DNA was included as a negative control. The cycling program consisted of 1×94° C. for 2 min.; 25×94° C. for 20 sec., 50° C. for 20 sec. and 72° C. for 45 sec.; and 1×72° C. for 2 min. After the completion of all cycles, 3 μl of 10× DNA loading buffer was added to each tube, and the amplified products were examined by 1.0% agarose gel electrophoresis. At least two replications were run on all reactions to ensure consistent results.

To confirm the amplified products from the L. monocytogenes strains were actually from the Imo0733 gene, the amplicons from nine of the strains (ATCC 19111, ATCC 19112, ATCC 19113, ATCC 19114, ATCC 19115, ATCC 19116, ATCC 19118, EGD, and ATCC 15313) were digested with EcoNI and BpmI. The digested PCR products were observed by agarose gel electrophoresis to determine if the digests yielded bands consistent with the predicted sizes based on the EGD genome sequence.

The specificity of the PCR assay was further assessed by Southern blot hybridization. Briefly, PCR products from three L. monocytogenes strains and seventeen other species were separated by agarose gel electrophoresis and transferred to a nylon membrane (Hybond N+, Amersham Pharmacia, Piscataway, N.J.) using a standard protocol (Ausubel, et al., Current Protocols in Molecular Biology, (1994)). The 453 bp Imo0733 amplicon from EGD was labelled using the ECL direct nucleic acid labelling and detection system (Amersham Pharmacia Biotech) and used to probe the membrane. Prehybridization, hybridization, and stringency washes were all performed in tubes at 42° C. according to the manufacturer's protocol.

The detection limit of the PCR assay was determined using the conditions described above with serial dilutions of the 10 ng/μl genomic DNA stock from L. monocytogenes EGD (NCTC 7973) as the template. DNA quantities tested consisted of 10 ng, 1 ng, 0.1 ng (100 pg), 0.01 ng (10 pg), 0.001 ng (1 pg) and 0.0001 ng (0.1 pg) per reaction. An equal volume (1.5 μl) of template DNA was added to each reaction.

Results.

After comparison of the genomes of L. innocua strain CLIP (serovar 6a) and L. monocytogenes EGD-e (serovar 1/2a), one L. monocytogenes specific gene (Imo0733) was selected. This gene is located between nucleotide sequences 123783-124307, and encodes a 169 amino acid protein similar to a transcriptional regulator (Glaser et al., Science, 294:849-852 (2001)). Two L. monocytogenes specific oligonucleotide primers (Imo0733F: 5′CGCAAGAAGAAATTGCCATC-3′ (SEQ ID NO:1) and Imo0733R: 5′-TCCGCGTTAGAAAAATTCCA-3′ (SEQ ID NO:2)) were designed from the coding sequence of this gene. These primers correspond to the /mo0733 gene sequences at nucleotide positions 123844-123863 and 124277-124296, respectively, and allow amplification of a 453 bp DNA fragment by PCR.

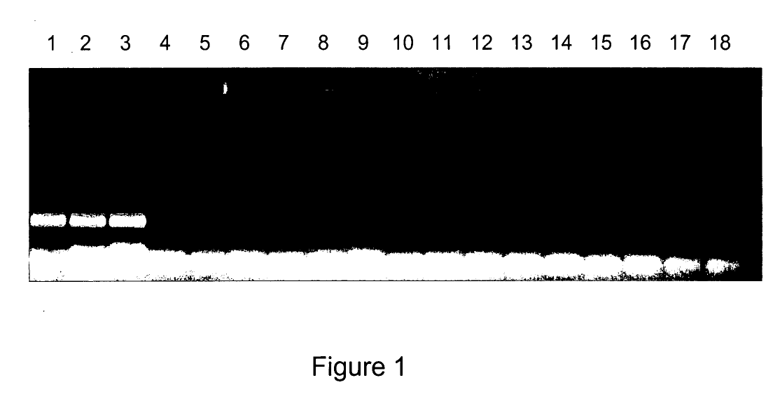

Using the L. monocytogenes specific primers (Imo0733F and Imo0733R), the predicted 453 bp fragment was amplified from genomic DNA from all 30 L. monocytogenes strains regardless of serotypes or origins (Table 1 and FIG. 1). DNA from species other than L. monocytogenes, as well as the negative control, did not produce detectable bands on ethidium bromide-stained agarose gels (Table 1 and FIG. 1). In FIG. 1 (an agarose gel electrophoresis), lane 1 a PCR product from genomic DNA of L. monocytogenes EGD; lane 2 is L. monocytogenes ATCC 15313; lane 3 is L. monocytogenes ATCC 19114; lane 4 is L. grayi ATCC 19120; lane 5 is L. grayi ATCC 25400; lane 6 is L. innocua ATCC 33090; lane 7 is L. innocua ATCC 43547; lane 8 is L. ivanovii ATCC 19119; lane 9 is L. ivanovii RM3325; lane 10 is L. seeligeri ATCC 35967; lane 11 is L. seeligeri RM3008; lane 12 is L. welshimeri ATCC 43550; lane 13 is L. welshimeri ATCC 43551; lane 14 is Clostridium perfringens; lane 15 is Escherichia coli ATCC 25922; lane 16 is Enterococcus faecalis ATCC 29212; lane 17 is Pseudomonas aeruginosa ATCC 27853; and lane 18 is no DNA control.

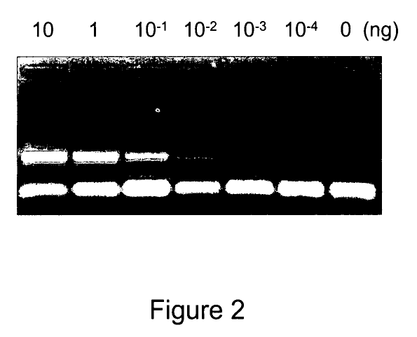

The detection threshold of these PCR primers under the described conditions using genomic DNA of L. monocytogenes grown in BHI broth was approximately 10 pg of DNA (FIG. 2). In FIG. 2, lane 1 is 10 ng of genomic DNA; lane 2 is 1 ng of genomic DNA; lane 3 is 0.1 ng (100 pg) of genomic DNA; lane 4 is 0.01 ng (10 pg) of genomic DNA; lane 5 is 0.001 ng (I pg) is of genomic DNA; lane 6, 0.0001 ng (0.1 pg) of genomic DNA; and lane 7 shows no DNA control.

The Imo0733 amplicons from nine of the L. monocytogenes strains were confirmed by digestion with EcoNI and BpmI. According to the L. monocytogenes EGD genome sequence, digestion with EcoNI should yield 322 bp and 131 bp fragments, and digestion with BpmI should yield 213 bp and 240 bp fragments. All of the amplicons from the nine strains yielded bands at the predicted sizes from both enzymes (data not shown).

Southern hybridization using the Imo0733 amplicon from strain EGD as a probe demonstrated that the PCR products amplified from ATCC 19114 and ATCC 15313 were from the same gene, and it also demonstrated that the Imo0733 PCR product was not detectable from other species, including other Listeria species (data not shown).

Taken together, these results show that application of PCR primers (Imo0733F and Imo0733R) derived from Imo0733 gene resulted in the amplification of a specific 453 bp fragment from L. monocytogenes DNA only, which suggests that this gene is not present in the other four Listeria species as well. This assay provides an alternative means of rapidly and precisely diagnosing listeriosis due to L. monocytogenes because it is based on a novel transcriptional regulator gene unique to L. monocytogenes instead of relying on rRNA genes, iap, or the virulence gene cluster.

In addition, these results suggest that Imo0733 may be an important virulence gene regulator that modulates expression of genes that allow L. monocytogenes to adapt to a human host.

TABLE 1
List of bacterial isolates examined by PCR using L. monocytogenes
specific primers

| | | | lmo0733 |
|---|---|---|---|
| Strain | Serovar | Source | (455 bp) |
| L. monocytogenes ATCC 19111 | 1 | Poultry | + |
| L. monocytogenes ATCC 19112 | 2 | Human | + |
| L. monocytogenes ATCC 19113 | 3 | Human | + |
| L. monocytogenes ATCC 19114 | 4a | Human | + |
| L. monocytogenes ATCC 19115 | 4b | Human | + |
| L. monocytogenes ATCC 19116 | 4c | Chicken | + |
| L. monocytogenes ATCC 19117 | 4d | Sheep | + |
| L. monocytogenes ATCC 19118 | 4e | Chicken | + |
| L. monocytogenes ATCC 15313 | 1 | Rabbit | + |
| L. monocytogenes EGD | ½a | Guinea pig | + |
| (NCTC7973) | | | |
| L. monocytogenes HCC7 | 1 | Catfish brain | + |
| L. monocytogenes HCC8 | 1 | Catfish brain | + |
| L. monocytogenes HCC12 | 4 | Catfish brain | + |
| L. monocytogenes HCC13 | 4 | Catfish kidney | + |
| L. monocytogenes HCC16 | 4 | Catfish brain | + |
| L. monocytogenes HCC17 | 4 | Catfish brain | + |
| L. monocytogenes HCC18 | 4 | Catfish spleen | + |
| L. monocytogenes HCC19 | 4 | Catfish spleen | + |
| L. monocytogenes HCC23 | 4 | Catfish brain | + |
| L. monocytogenes HCC24 | 4 | Catfish spleen | + |
| L. monocytogenes HCC25 | 4 | Catfish kidney | + |
| L. monocytogenes 168 | | Aborted calf | + |
| | | fetus | |
| L. monocytogenes 180 | | Human outbreak | + |
| L. monocytogenes 418 | | Freezer study | + |
| L. monocytogenes 742 | | Ground beef | + |
| L. monocytogenes 874 | | Cow brain | + |
| L. monocytogenes 1002 | | Pork sausage | + |
| L. monocytogenes 1084 | | Chicken | + |
| L. monocytogenes 1400 | | CDC/Jalisco | + |
| | | outbreak | |
| L. monocytogenes CWD 1028 | ½c | Pork sausage | + |
| L. innocua ATCC 33090 | 6a | Cow brain | − |
| L. innocua ATCC 43547 | 6b | Bovine brain | − |
| L. innocua 415 | | Turkey burger | − |
| L. innocua 416 | | Veal/beef patty | − |
| L. innocua 417 | | Beef steak | − |
| L. innocua 662 | | Raw milk | − |
| L. innocua 1419 | | Ground cheese | − |
| L. innocua 1425 | | Pecorino | − |
| | | Romano | |
| L. innocua 1720 | | Chicken | − |
| L. innocua 1944 | | Ground turkey | − |
| L. grayi ATCC 19120 | | Chinchilla | − |
| | | faeces | |
| L. grayi ATCC 25400 | | Corn | − |
| | | leaves/stalks | |
| L. grayi (murrayi) ATCC 25401 | | Corn | − |
| | | leaves/stalks | |
| L. ivanovii ATCC 19119 | | Sheep | − |
| L. ivanovii RM3325 | | Cheese | − |
| L. seeligeri ATCC 35967 | | Soil | − |
| L. seeligeri RM3008 | | Soil | − |
| L. seeligeri RM3321 | | Cheese | − |
| L. welshimeri ATCC 35897 | | Plant | − |
| L. welshimeri ATCC 43550 | ½b | Soil | − |
| L. welshimeri ATCC 43551 | 6a | Soil | − |
| L. welshimeri CCF4 | | Catfish brain | − |
| L. welshimeri 1471 | | Environment | − |
| Aeromonas hydrophila ATCC | | | − |
| 35654 | | | |
| Clostridium perfringens | | Clinical | − |
| Enterococcus faecalis ATCC | | | − |
| 29212 | | | |
| Escherichia coli ATCC 25922 | | | − |
| Flavobacterium indolegenes | | Clinical | − |
| Klebsiella pneumoniae ATCC | | | − |
| 13883 | | | |
| Proteus vulgaris ATCC 13315 | | | − |
| Pseudomonas aeruginosa ATCC | | | − |
| 27853 | | | |
| Salmonella typhimurium ATCC | | | − |
| 14028 | | | |
| Serratia marcescens ATCC | | | − |
| 8100 | | | |
| Staphylococcus aureus ATCC | | | − |
| 25923 | | | |
| Streptococcus pneumoniae | | Clinical | − |
| Streptococcus pyogenes ATCC | | | − |
| 19615 | | | |
| Vibrio cholerae | | Clinical | − |
| Yersinia pseudotuberculosis | | Clinical | − |

### EXAMPLE 2

### Specific PCR Identification of Pasteurella multocida Based on Putative Transcriptional Regulator Genes

Bacteria.

A collection of 45 bacterial strains/isolates, including 10 Pasteurella multocida, 5 Mannheimia haemolytica, and 30 other bacterial species, were analysed (Table 2). These bacteria were either acquired from the American Type Culture Collection (ATCC) or the National Collection of Type Culture (NCTC), or isolated from clinical samples in our laboratory. Bacterial strains were initially grown on 5% sheep blood agar plates (TSA II, Becton Dickinson Microbiology Systems, Cockeysville, Md.) and transferred to flasks containing 25 ml of brain heart infusion (BHI) broth for batch cultures (Difco Laboratories, Detroit, Mich.). Bacterial cultures were incubated at 37° C. overnight with rotary aeration.

Preparation of Bacterial DNA.

Bacterial DNA was prepared using the method described in Example 1.

Identification of P. multocida Specific Transcriptional Regulator Genes.

The nucleotide sequences of P. multocida genes encoding transcriptional regulators were retrieved from the published P. multocida genome sequence (May et al, 2001), and BLAST searches were conducted to select those transcriptional regulator genes that display no homology with other DNA sequences at GenBank. Oligonucleotide primers were designed from these genes with Primer3 software (Whitehead Institute for Medical Research, Cambridge, Mass.) and synthesized by Sigma Genosys (The Woodlands, Tex.).

PCR Amplification.

PCR amplification was conducted in a volume of 25 μl using a GeneAmp PCR System 2400 (Perkin Elmer, Foster City, Calif.). The reaction mixture consisted of 0.5 U Taq DNA polymerase (Fisher Scientific, Houston, Tex.), 1× PCR buffer (10 mM Tris-HCl pH 9.0, 50 mM KCl and 1.5 mM MgCl₂), 50 μM dNTPs, 25 pmol primers each and 15 ng DNA. Reaction mixture with no template DNA was used as a negative control. The cycling programs consisted of 1 cycle of 94° C. for 2 min.; 25 cycles of 94° C. for 20 sec., 60° C for 20 sec. and 72° C. for 45 sec.; and a final incubation at 72° C. for 2 min. After completion of all cycles, 3 μl of 10× DNA loading buffer was added to each tube, and the amplified products were examined by 1.0% agarose gel electrophoresis in the presence of ethidium bromide (0.5 μg/ml). The stained gels were visualized under UV light, and results were recorded using a Chemilmager 5500 (BSI, Stafford, Tex.).

Results.

Upon comparison of P. multocida transcriptional regulator gene sequences (May et al, 2001) with other DNA sequences at GenBank via BLAST searches, two genes (Pm0762 and Pm1135) with no apparent homology to published DNA sequences were selected for further evaluation (Table 3). Oligonucleotide primers Pm0762F (SEQ ID NO:3) and Pmo0762R (SEQ ID NO:4) were designed to amplify a 567 bp DNA fragment by PCR, and primers Pm1135F (SEQ ID NO:5) and Pm1135R (SEQ ID NO:6) were designed to produce 489 bp product (Table 3).

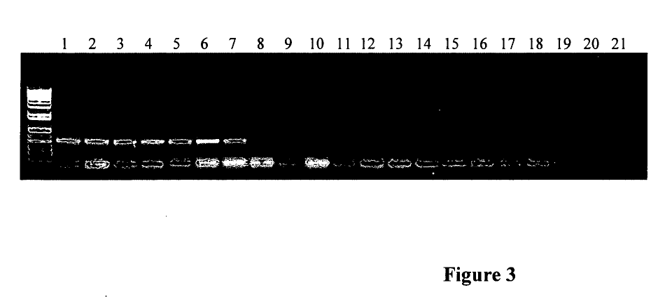

The specificity of these primers was assessed with a collection of 45 bacterial strains/isolates, including 10 P. multocida, 5 M. haemolytica, and 30 other bacterial species (Table 2). As expected, both primer sets formed specific PCR products of appropriate size from genomic DNA of P. multocida only, but not from DNA of other bacterial species (Table 2 and FIG. 3). In FIG. 3, lanes 1-7 contain amplified DNA products from Pasturella multocida strains (ATCC 11039, x-3422, x-1623, x-1682, w-8434, w-8531 and w-8267); lanes 8-12 are Mannheimia haemolytica strains (D139, D144, D153, D180 and 55518); lane 1 is SaImonella typhimurium ATCC 14028; lane 14 is Staphylococcus aureus ATCC 25923; lane 15 is Streptococcus pyogenes ATCC 19615; lane 16 is L. innocua ATCC 33090; lane 17 is L. ivanovii ATCC 19119; lane 18 is L. monocytogenes EGD (NCTC 7973); lane 19 is L. seeligeri ATCC 35967; lane 20 is L. welshimeri ATCC 35897; and lane 21 is the negative control with no template DNA. On the left of lane 1 is DNA molecular weight marker (1 kb plus DNA ladder, Invitrogen, Carlsbad, Calif.).

These results show that Pm0762 and Pm1135 are species-specific genes in P. multocida. The application of these primers (Pm0762F/R and Pm1135F/R) would therefore offer an additional means of rapidly and precisely identifying P. multocida.

TABLE 2
List of bacterial strains examined by PCR using primers from P. multocida putative
transcriptional regulator genes (Pm0762 and Pm1135)

| Strain | Source | Pm0762(567 bp) | Pm1135(489 bp) |
|---|---|---|---|
| Pasteurella multocida ATCC 11039 | | + | + |
| P. multocida x3422 | Cat wound | + | + |
| P. multocida x-1623 | Pig thorax | + | + |
| P. multocida x-1682 | Pig lung | + | + |
| P. multocida w-8434 | Pig lung | + | + |
| P. multocida w-8531 | Pig lung | + | + |
| P. multocida w-8267 | Bovine milk | + | + |
| P. multocida w-6984 | | + | + |
| P. multocida w-7069 | | + | + |
| P. multocida w-7858 | | + | + |
| Mannheimia haemolytica D139 | | − | − |
| M. haemolytica D144 | | − | − |
| M. haemolytica D153 | | − | − |
| M. haemolytica D180 | | − | − |
| M. haemolytica 55518 | | − | − |
| Aeromonas hydrophila ATCC 35654 | | − | − |
| Clostridium perfringens | Clinical | − | − |
| Enterococcus faecalis ATCC 29212 | | − | − |
| Enterococcus faecalis 844-4 | Clinical | − | − |
| Escherichia coli ATCC 25922 | | − | − |
| Flavobacterium indolegenes | Clinical | − | − |
| Klebsiella pneumoniae ATCC 13883 | | − | − |
| Listeria grayi ATCC 25400 | Corn leaves/stalks | − | − |
| L. murrayi ATCC 25401 | Corn leaves/stalks | − | − |
| L. innocua ATCC 33090 | Cow brain | − | − |
| L. innocua CLIP 11262 | | − | − |
| L. ivanovii ATCC 19119 | Sheep | − | − |
| L. ivanovii SLCC 6965 | AH | − | − |
| L. monocytogenes EGD (NCTC 7973) | Guinea pig | − | − |
| L. monocytogenes ATCC 15313 | Rabbit | − | − |
| L. monocytogenes HCC23 | Catfish brain | − | − |
| L. seeligeri ATCC 35967 | Soil | − | − |
| L. seeligeri SLCC 5921 | AH | − | − |
| L. welshimeri ATCC 35897 | Plant | − | − |
| L. welshimeri ATCC 43550 | Soil | − | − |
| Proteus vulgaris ATCC 13315 | | − | − |
| Pseudomonas aeruginosa ATCC 27853 | | − | − |
| Salmonella typhimurium ATCC 14028 | | − | − |
| Serratia marcescens ATCC 8100 | | − | − |
| Staphylococcus aureus ATCC 25923 | | − | − |
| Streptococcus pyogenes ATCC 19615 | | − | − |
| S. pneumonia | Clinical | − | − |
| S. uberis 815 | Clinical | − | − |
| S. zooepidemicus 865-2 | Clinical | − | − |
| Yersinia pseudotuberculosis | Clinical | − | − |

TABLE 3

Oligonucleotide primers of P. multocida putative
transcriptional regulator genes (Pm0762 and Pm1135)

| | | | | PCR | |
|---|---|---|---|---|---|
| | | | | Product | Primer |
| Gene | Nucleotides | Forward (5′-3′) | Reverse (5′-3′) | (bp) | positions |
| Pm0762 | 267-1013 | Ttgtgcagttccgcaaataa | Ttcacctgcaacagcaagc | 567 | 355-374; |
| | | (SEQ ID NO:3) | (SEQ ID NO:4) | | 921-902 |
| Pm1135 | 7167-7730 | ccatgtccgttgaatcacag | atggcgaaagaagggttaca | 489 | 7185-7204; |
| | | (SEQ ID NO:5) | (SEQ ID NO:6) | | 7673-7654 |

### EXAMPLE 3

### Evaluation of PCR Primers from Putative Transcriptional Regulator Genes for Identification of Staphylococcus aureus

Bacteria.

Bacterial reference strains were acquired from the American Type Culture Collection (ATCC) and the National Collection of Type Culture (NCTC). Other strains were either isolated in this laboratory from clinical samples or obtained from external sources. A collection of 63 bacterial strains/isolates were analysed in this study. These included 14 S. aureus, 3 other Staphylococcus species, and 46 other bacterial species (Table 4). Bacterial strains were cultivated on 5% sheep blood agar plates (TSA II, Becton Dickinson Microbiology Systems, Cockeysville, Md.), and batch cultures were grown in of brain heart infusion (BHI) broth (Difco Laboratories, Detroit, Mich.) at 37° C. with rotary aeration. Extraction of bacterial DNA.

Bacterial DNA was prepared using the method described in Example 1.

Identification of S. aureus Specific Transcriptional Regulator Genes

The nucleotide sequences of S. aureus genes encoding transcriptional regulators were retrieved from the published genome sequences of S. aureus (Kuroda et al, 2001), and BLAST searches were conducted to select transcriptional regulator genes demonstrating no homology with other gene sequences in GenBank. Oligonucleotide primers were designed from the transcriptional regulator genes unique to S. aureus with Primer3 software (Whitehead Institute for Medical Research, Cambridge, Mass.) and synthesized by Sigma Genosys (The Woodlands, Tex.).

PCR Amplification.

PCR amplification was performed in a volume of 25 μl using a GeneAmp PCR System 2400 (Perkin Elmer, Foster City, Calif.). The reaction mixture consisted of 0.5 U Taq DNA polymerase (Fisher Scientific, Houston, Tex.), 1× PCR buffer (10 mM Tris-HCl pH 9.0, 50 mM KCl and 1.5 mM MgCl₂), 50 μM dNTPs, 25 pmol primers each and 15 ng DNA. Reaction mixture with no template DNA was used as a negative control. The cycling programs consisted of 1 cycle of 94° C. for 2 min.; 25 cycles of 94° C. for 20 sec., 60° C. for 20 sec. and 72° C. for 45 sec.; and a final incubation at 72° C. for 2 min. After completion of all cycles, 3 μl of 10× DNA loading buffer was added to each tube, and the amplified products were examined by 1.0% agarose gel electrophoresis in the presence of ethidium bromide (0.5 μg/ml). The stained gels were visualized under UV light and results recorded using a Chemilmager 5500 (BSI, Stafford, Tex.).

Results.

After comparison of S. aureus transcriptional regulator genes (Kuroda et al, 2001) with other DNA sequences at GenBank via BLAST searches, two genes (i.e., Sa0836 and Sa0856) that encode putative transcriptional regulators were selected for further evaluation (Table 5). These genes appeared to be unique because they displayed no apparent homology with previously published DNA sequences. Oligonucleotide primers (Sa0836F (SEQ ID NO:7) and Sa0836R (SEQ ID NO:8)) were derived from Sa0836 that facilitated amplification of a 573 bp amplicon, and primers derived from Sa0856 (Sa0856F (SEQ ID NO:9) and Sa0856R (SEQ ID NO:10)) yielded a band of 599 bp by PCR (Table 5).

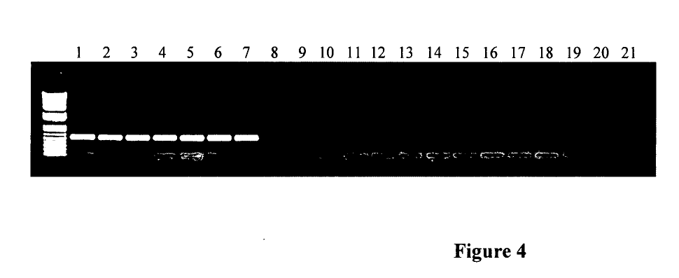

The specificity of these primers was evaluated with a collection of 63 bacterial strains/isolates containing 14 S. aureus, 3 other Staphylococcus and 46 other species (Table 4). Primers derived from these two genes generated PCR products of expected sizes from genomic DNA of S. aureus only, and not from DNA of other bacterial species (FIG. 4 and Table 4). In FIG. 4, both panels, lanes 1-7 contain PCR products amplified from Staphylococcus aureus strains (ATCC 25923, 814, 815, 819, 820, 82 1and 836); lanes 8-10 are Staphylococcus species (818, 822, and 823); lane 11 is Streptococcus dysgalactiae 814; lane 12 is S. equinus 866; lane 13 is S. equisimilis 834-4; lane 14 is S. intermedius 845; lane 15 is S. pneumoniae; lane 16 is S. pyogenes ATCC 19615; lane 17 is S. uberis 815; lane 18 is S. zooepidemicus 861-1; lane 19 is L. innocua ATCC 33090; lane 20 is L. monocytogenes EGD (NCTC 7973); and lane 21 is negative control with no template DNA. On the left of lane 1, it contains DNA molecular weight marker (1 kb plus DNA ladder, Invitrogen, Carlsbad, Calif.).

These results indicate that S. aureus genes (Sa0836 and Sa0856) encoding putative transcriptional regulators are species-specific.

TABLE 4
List of bacterial strains examined by PCR using primers from S. aureus
putative transcriptional regulator genes (Sa0836 and Sa0856)

| | | Sa0836 | Sa0856 |
|---|---|---|---|
| Strain | Source | (573 bp) | (599 bp) |
| Staphylococcus aureus ATCC 25923 | | + | + |
| S. aureus 814 | Clinical | + | + |
| S. aureus 815 | Clinical | + | + |
| S. aureus 819 | Clinical | + | + |
| S. aureus 820 | Clinical | + | + |
| S. aureus 821 | Clinical | + | + |
| S. aureus 836 | Clinical | + | + |
| S. aureus 838 | Clinical | + | + |
| S. aureus 1037 | Clinical | + | + |
| S. aureus 1046 | Clinical | + | + |
| S. aureus 1063 | Clinical | + | + |
| S. aureus 1102 | Clinical | + | + |
| S. aureus 1152 | Clinical | + | + |
| S. aureus 1155 | Clinical | + | + |
| Staphylococcus spp. 818 | Clinical | − | − |
| Staphylococcus spp. 822 | Clinical | − | − |
| Staphylococcus spp. 823 | Clinical | − | − |
| Streptococcus canis 1001-3 | Clinical | − | − |
| S. dysgalactiae 814 | Clinical | − | − |
| S. dysgalactiae 822 | Clinical | − | − |
| S. dysgalactiae 824 | Clinical | − | − |
| S. equinus | Clinical | − | − |
| S. equinus 866 | Clinical | − | − |
| S. equisimilis 834-4 | Clinical | − | − |
| S. equisimilis 888-2 | Clinical | − | − |
| S. intermedius 845 | Clinical | − | − |
| S. intermedius 840 | Clinical | − | − |
| S. intermedius 1083 | Clinical | − | − |
| S. intermedius 1095 | Clinical | − | − |
| S. pneumoniae | Clinical | − | − |
| S. pyogenes ATCC 19615 | | − | − |
| S. uberis 815 | Clinical | − | − |
| S. uberis 820 | Clinical | − | − |
| S. zooepidemicus 861-1 | Clinical | − | − |
| S. zooepidemicus 861-2 | Clinical | − | − |
| S. zooepidemicus 865-2 | Clinical | − | − |
| S. zooepidemicus 897-1 | Clinical | − | − |
| S. zooepidemicus 1074-1 | Clinical | − | − |
| Aeromonas hydrophila ATCC 35654 | | − | − |
| Clostridium perfringens | Clinical | − | − |
| Enterococcus faecalis ATCC 29212 | | − | − |
| Enterococcus faecalis 844-4 | Clinical | − | − |
| Escherichia coli ATCC 25922 | | − | − |
| Flavobacterium indolegenes | Clinical | − | − |
| Klebsiella pneumoniae ATCC 13883 | | − | − |
| Listeria grayi ATCC 25400 | Corn | − | − |
| | leaves/ | | |
| | stalks | | |
| L. murrayi ATCC 25401 | Corn | − | − |
| | leaves/ | | |
| | stalks | | |
| L. innocua ATCC 33090 | Cow brain | − | − |
| L. innocua CLIP 11262 | | − | − |
| L. ivanovii ATCC 19119 | Sheep | − | − |
| L. ivanovii SLCC 6965 | AH | − | − |
| L. monocytogenes EGD (NCTC | Guinea | − | − |
| 7973) | pig | | |
| L. monocytogenes ATCC 15313 | Rabbit | − | − |
| L. monocytogenes HCC23 | Catfish | − | − |
| | brain | | |
| L. seeligeri ATCC 35967 | Soil | − | − |
| L. seeligeri SLCC 5921 | AH | − | − |
| L. welshimeri ATCC 35897 | Plant | − | − |
| L. welshimeri ATCC 43550 | Soil | − | − |
| Proteus vulgaris ATCC 13315 | | − | − |
| Pseudomonas aeruginosa ATCC | | − | − |
| 27853 | | | |
| Salmonella typhimurium ATCC | | − | − |
| 14028 | | | |
| Serratia marcescens ATCC 8100 | | − | − |
| Yersinia pseudotuberculosis | Clinical | − | − |

TABLE 5

Oligonucleotide primers derived from putative
transcriptional regulator genes of S. aureus

| | | | | PCR | |
|---|---|---|---|---|---|
| | | | | Product | Primer |
| Gene | Nucleotides | Forward (5′-3′) | Reverse (5′-3′) | (bp) | positions |
| Sa0836 | 51312-52181 | ggcgcttgtaaaattttcgt | tgcgcaaagttttattgaaca | 573 | 51409-51429; |
| | | (SEQ ID NO:7) | (SEQ ID NO:8) | | 51981-51961 |
| Sa0858 | 74441-75427 | ctgtgtgtggcaagcaagtt | ttgccagcgacaatatttga | 599 | 74517-74536; |
| | | (SEQ ID NO:9) | (SEQ ID NO:10) | | 75115-75096 |

### EXAMPLE 4

### Rapid Identification of Streptococcus pyogenes with PCR Primers from a Putative Transcriptional Regulator Gene

Bacterial Strains.

A collection of 60 bacterial strains comprising 10 Streptococcus pyogenes, 16 non-pyogenes streptococci, and 34 other Gram-positive and -negative bacteria was examined in the study (Table 6). Of these, the reference strains were obtained from the American Type Culture Collection (ATCC) and the National Collection of Type Culture (NCTC); Streptococcus pyogenes human isolates were originated from Department of Microbiology, University of Alabama at Birmingham, Ala.; Staphylococcus aureus human isolates from Veterans Affairs Medical Center, Jackson, Miss.; other clinical/food bacterial strains were mostly isolated at College of Veterinary Medicine, Mississippi State University, Mississippi State, Miss. except for Mannheimia haemolytica D139 that was provided by Dr. Robert Briggs of National Animal Disease Center, Ames, Iowa.

Genomic DNA.

Bacterial DNA was prepared using the method described in Example 1.

Identification of S. pyogenes Specific Gene(s).

S. pyogenes genes that encode transcriptional regulators were obtained from the genome sequence of a M1 GAS strain SF370 (Ferretti, et al., Proc. Natl. Acad. Sci. USA, 98:4658:4653 (2001)), and screened against other DNA sequences at GenBank by BLAST searches. Only gene(s) uniquely present in S. pyogenes were selected for further evaluation. Oligonucleotide primers were then designed from the gene(s) of interest with Primer3 software (Whitehead Institute for Medical Research, Cambridge, Mass.), and synthesized by Sigma Genosys (The Woodlands, Tex.).

PCR Amplification.

PCR was conducted in a 25 μl volume using a GeneAmp PCR System 9600 (Perkin Elmer). The reaction mixture (25 μl) comprised 0.5 U Taq DNA polymerase (Fisher Scientific, Houston, Tex.), 1× PCR buffer (containing 10 mM Tris-HCI pH 9.0, 50 mM KCl and 1.5 mM MgCl₂), 50 μM dNTPs, 25 pmol primers each and 10 ng DNA. The reaction mixture with no template DNA was used as a negative control. The cycling programs consisted of 1×94° C. for 2 min., 30×94° C. for 20 sec., 55° C. for 20 sec. and 72° C. for 45 sec., and 1×72° C. for 2 min. fter completion of all cycles, 3 μl of 10× DNA loading buffer was added to each tube, and the amplified products were examined in 1.0% agarose gel electrophoresis in the presence of ethidium bromide (0.5 μg/ml). The stained gels were visualized under UV light and photographed using a Chemilmager 5500 (BSI, Stafford, Tex.).

Based on the results form BLAST searches, a S. pyogenes specific gene (Spy1258) that encodes a putative transcriptional regulator was identified from the genome sequence of a M1 strain SF370 (GenBank accession No. AE006565) (Ferretti, et al., Proc. NatL Acad. Sci. USA, 98:4658-4663 (2001)). It was noted that a stretch of nucleotides identical to Spy1258 (nt. 6651-7193) was also found in the complete genomes of S. pyogenes M3 strains MGAS315 (GenBank accession No. AE014154) (Beres, et al., Proc. Natl. Acad. Sci. USA, 99:10078-10083 (2002) and SSI-1 (GenBank accession No. AP005144) (Nakagawa, et al. Genome Res., 13:1042-1055 (2003)) as well as M18 strain MGAS8232 (GenBank accession No. AE010045) Smoot, et al., Proc. Natl. Acad. Sci. USA, 99:4668-4673 (2002)). However, the Spy1258 gene sequence was clearly absent in other bacterial genomes that are available at GenBank.

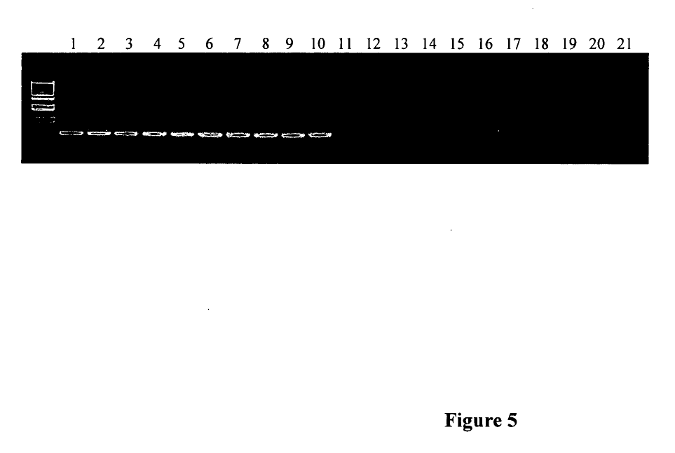

Therefore, a pair of oligonucleotide primers was designed from the putative transcriptional regulator gene Spy1258 (i.e., spy1258F: 5′-AAAGACCGCCTTAACCACCT-3′ (SEQ ID NO:11) and spy1258R: 5′-TGGCAAGGTAAACTTCTAAAGCA-3′ (SEQ ID NO:12)). These primers correlate to the Spy1258 gene sequence at nt 6686-6705 and nt 7092-7070, respectively, which facilitate the amplification of a 407 bp DNA fragment from S. pyogenes. Using these primers (spy1258F and spy1258R) in PCR with a collection of 60 bacterial strains, it was observed that a specific DNA fragment of the expected size (407 bp) was generated from all ten S. pyogenes strains only, but not from 16 non-pyogenes Streptococci (representing 7 separate species) and 34 other bacteria (Table 6 and FIG. 5). The accompanying negative (no DNA template) control did not yield any amplification product with these primers either (FIG. 5). In FIG. 5, lanes 1-10, are the PCR products amplified from genomic DNA of Streptococcus pyogenes (ATCC 19615, M1, M2, M3, M12, M14, M22, M28, T9 and T9/5/27/44); lane 11 is S. canis 1001-3; lane 12 is S. dysgalactiae 814; lane 13 is S. eqinus 866; lane 14 is S. equisimilis 824-4; lane 15 is S. pheumoniae; lane 16, S. uberis 815; lane 17 is S. zooepidemicus 861-1; lane 18 is Staphylococcus aureus ATCC 25923; lane 19 is L. monocytogenes EGD (NCTC 7973); lane 20 is E. faecalis ATCC 29212; and lane 21 is the control with no DNA template. The left of lane 1 was DNA molecular marker (1 Kb Plus DNA Ladder, Life Technologies).

These results show that the putative transcriptional regulator gene Spy1258is specific for S. pyogenes, and it can be used as a diagnostic marker for rapid confirmation of Group A streptococci.

TABLE 6
List of bacterial strains examined in this study

| | | Spy1258 |
|---|---|---|
| Strain | Source | (407 bp) |
| Streptococcus pyogenes ATCC 19615 | Human pharynx | + |
| S. pyogenes MGAS166 (JM007) M1 | Human | + |
| S. pyogenes MGAS327 (JM048) M2 | Human | + |
| S. pyogenes MGAS159 (JM008) M3 | Human | + |
| S. pyogenes MGAS278 (JM006) M12 | Human | + |
| S. pyogenes MGAS321 (JM002) M14 | Human | + |
| S. pyogenes MGAS162 (JM041) M22 | Human | + |
| S. pyogenes MGAS325 (JM001) M28 | Human | + |
| S. pyogenes MGAS280 (JM010) T9 | Human | + |
| S. pyogenes MGAS296 (JM033) | Human | + |
| T9/5/27/44 | | |
| S. canis 1001-3 | Clinical | − |
| S. dysgalactiae 814 | Milk | − |
| S. dysgalactiae 822 | Milk | − |
| S. dysgalactiae 824 | Milk | − |
| S. equinus | Horse | − |
| S. equinus 866 | Horse | − |
| S. equisimilis 834-4 | Clinical | − |
| S. equisimilis 888-2 | Clinical | − |
| S. pneumoniae | Clinical | − |
| S. uberis 815 | Clinical | − |
| S. uberis 820 | Clinical | − |
| S. zooepidemicus 861-1 | Horse | − |
| S. zooepidemicus 861-2 | Horse | − |
| S. zooepidemicus 865-2 | Horse | − |
| S. zooepidemicus 897-1 | Horse | − |
| S. zooepidemicus 1074-1 | Horse | − |
| Staphylococcus aureus ATCC 25923 | Clinical | − |
| S. aureus 814 | Milk | − |
| S. aureus 815 | Milk | − |
| S. cohnii 4850 | Chicken | − |
| S. epidermidis 979 | Human | − |
| S. epidermidis 986 | Human | − |
| S. haemolyticus 2168 | Human | − |
| S. hominus 1961 | Human | − |
| S. hominus 2130 | Human | − |
| S. Saccharolyticus 956 | Human | − |
| S. intermedius 840 | Dog | − |
| S. intermedius 845 | Dog | − |
| S. intermedius 1083 | Dog | − |
| S. siuri 4856 | Squirrel brain | − |
| Aeromonas hydrophila ATCC 35654 | | − |
| Bordetella avium 2-1178 | | |
| B. bronchiseptica | Pig | |
| Clostridium perfringens | Clinical | − |
| Enterococcus faecalis ATCC 29212 | Urine | − |
| E. faecalis 844-4 | Clinical | − |
| Escherichia coli ATCC 25922 | Clincal | − |
| Flavobacterium indolegenes | Clinical | − |
| Klebsiella pneumoniae ATCC 13883 | | − |
| Listeria grayi ATCC 25400 | Corn leaves/stalks | − |
| L. grayi ATCC 25401 | Corn leaves/stalks | − |
| L. innocua ATCC 33090 | Cow brain | − |
| L. innocua CLIP 11262 | | − |
| L. ivanovii ATCC 19119 | Sheep | − |
| L. ivanovii SLCC 6965 | FDA | − |
| L. monocytogenes EGD (NCTC 7973) | Guinea pig | − |
| L. monocytogenes ATCC 15313 | Rabbit | − |
| L. monocytogenes HCC23 | Catfish brain | − |
| L. seeligeri ATCC 35967 | Soil | − |
| L. seeligeri SLCC 5921 | FDA | − |
| L. welshimeri ATCC 35897 | Plant | − |
| L. welshimeri ATCC 43550 | Soil | − |
| Mannheimia haemolytica D139 | Bovine lung | − |
| Micrococcus sedentarius 2134 | Human | − |
| Pasteurella multocida ATCC 11039 | Domestic fowl | − |
| Proteus vulgaris ATCC 13315 | | − |
| Pseudomonas aeruginosa ATCC 27853 | Blood culture | − |
| Salmonella typhimurium ATCC 14028 | | − |
| Serratia marcescens ATCC 8100 | | − |
| Yersinia pseudotuberculosis | Clinical | − |

### EXAMPLE 5

### PCR Amplification of A Species-Specific, Putative Transcriptional Regulator Gene Reveals the Identity of Enterococcus faecalis

Bacterial Strains.

A collection of 88 bacterial strains including 22 E. faecalis, 11 E. faecium and 55 other Gram-positive and -negative bacteria were examined in the study (Table 7). Of these, the reference strains were obtained from the American Type Culture Collection (ATCC) and the National Collection of Type Culture (NCTC); E. faecalis and E. faecium were cultured from seafood; and other bacteria were isolated from clinical specimens of human and animal origins.

Genomic DNA.

Bacterial DNA was prepared using the method described in Example 1.

Identification of E. faecalis Specific Gene(s).

E. faecalis genes that encode transcriptional regulators were retrieved from the genome sequence of a vancomycin-resistant E. faecalis strain V583 (Paulsen, I. T., et al., 2003, Science 299, 2071-2074) and screened against other DNA sequences at GenBank by BLAST searches. Only E. faecalis genes showing no obvious homology with other DNA sequences were selected for further evaluation. Oligonucleotide primers were then designed from the gene(s) of interest with Primer3 software (Whitehead Institute for Medical Research, Cambridge, Mass.), and synthesized by Sigma Genosys (The Woodlands, Tex.).

PCR Amplification.

PCR was performed in a 25 μl volume using a GeneAmp PCR System 9600 (Perkin Elmer). The reaction mixture (25 μl) consisted of 0.5 U Taq DNA polymerase (Fisher Scientific, Houston, Tex.), 1× PCR buffer (containing 10 mM Tris-HCl pH 9.0, 50 mM KCl and 1.5 mM MgCl₂), 50 μM dNTPs, 25 pmol each forward and reverse primers and 10 ng DNA. The reaction mixture with no template DNA was included as a negative control. The cycling programs consisted of 1×9° C. for 2 min., 30×94° C. for 20 sec., 60° C. for 20 sec. and 72° C. for 45 sec., and 1×72° C. for 2 min. After completion of all cycles, 3 μL of 10× DNA loading buffer was added to each tube, and the PCR products were examined in 1.0% agarose gel electrophoresis in the presence of ethidium bromide (0.5 μg/ml). The stained gels were then visualized under UV light and photographed by using a Chemiumager 5500 (BSI, Stafford, Tex.).

Results.

After comparison of E. faecalis transcriptional regulator genes with other DNA sequences at GenBank via BLAST searches, an E. faecalis specific gene (Ef0027, nucleotides 27614-28384) that encodes a putative phosphosugar-binding transcriptional regulator was selected from the sequence data of a vancomycin-resistant E. faecalis strain V583 (GenBank accession No. AF454824) (Paulsen, et al., Science, 299:2071-2974 (2003)). This gene appeared to be uniquely present in E. faecalis as it showed no homology with other microbial genomes that are available at GenBank. Therefore, forward and reverse oligonucleotide primers were designed from this gene (i.e., Ef0027F: 5′-GCCACTATTTCTCGGACAGC-3′ (SEQ ID NO:13) and Ef0027R: 5′-GTCGTCCCTTTGGCAAATAA-3′ (SEQ ID NO:14)). These primers correspond to the Ef0027 gene sequence at nt 27777-27786; and nt 28284-28265, respectively, which enable the production of a 518 bp fragment from E. faecalis DNA in PCR.

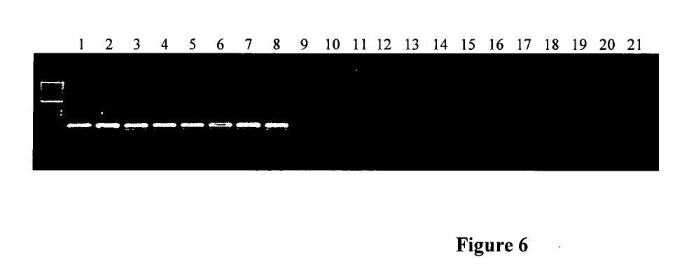

The specificity of the E. faecalis specific primers from the putative transcriptional regulator gene Ef0027 (i.e., Ef0027F and Ef0027R) was evaluated in PCR with a collection of 88 bacterial strains, including 22 E. faecalis, 11 E. faecium and 55 other Gram-positive and -negative bacteria (Table 7). It appeared that a specific DNA fragment of the expected size (518 bp) was amplified from E. faecalis strains only, but not from E. faecium and other bacteria as well as no DNA template control (Table 7 and FIG. 6). In FIG. 6, lanes 1-8 contain PCR products from genomic DNA of Enterococcus faecalis strains (ATCC 376, B4-1, B4-7, B4-9, B4-15, B4-17, B4-33 and B435); lane 9 is E. faecium ATCC 349; lane 11 is Streptococcus pyogenes ATCC 19615; lane 12 is Staphyloccoccus aureus ATCC 25923; lane 13 is Listeria monocytogenes EGD; lane 14 is L. grayi ATCC 25400; lane 15 is L. innocua ATCC 33090; lane 16 is L. ivanovii ATCC 19119; lane 17 is L. seeligeri ATCC 35967; lane 18 is L. welshimeri ATCC 35897; lane 18 is Pasteurella inultocida ATCC 11039; lane 19 is Pseudomonas aeruginosa ATCC 27853; lane 20 is Salmonella typhimurium ATCC 14028; and lane 21 is a control with no DNA templete. The left of lane 1 is DNA molecular marker (1 Kb DNA Ladder, Life Technologies). These results show that the putative transcriptional regulator gene Ef0027 is specific for E. faecalis, and it can be useful as a diagnostic marker for this bacterium.

TABLE 7
List of bacterial strains that have been examined

| | | Ef0027 |
|---|---|---|
| Strain | Source | (518 bp) |
| Enterococcus faecalis ATCC 29212 | | + |
| E. faecalis ATCC 376 | | + |
| E. faecalis B4-1 | Seafood | + |
| E. faecalis B4-7 | Seafood | + |
| E. faecalis B4-9 | Seafood | + |
| E. faecalis B4-13 | Seafood | + |
| E. faecalis B4-15 | Seafood | + |
| E. faecalis B4-17 | Seafood | + |
| E. faecalis B4-33 | Seafood | + |
| E. faecalis B4-35 | Seafood | + |
| E. faecalis B4-43 | Seafood | + |
| E. faecalis B4-63 | Seafood | + |
| E. faecalis B4-65 | Seafood | + |
| E. faecalis B4-67 | Seafood | + |
| E. faecalis B4-77 | Seafood | + |
| E. faecalis B5-39 | Seafood | + |
| E. faecalis B5-43 | Seafood | + |
| E. faecalis B 5-49 | Seafood | + |
| E. faecalis B5-51 | Seafood | + |
| E. faecalis B5-61 | Seafood | + |
| E. faecalis 844-4 | Seafood | + |
| E. faecalis 1085 | Seafood | + |
| E. faecium ATCC 349 | | − |
| E. faecium B2-1 | Seafood | − |
| E. faecium B2-3 | Seafood | − |
| E. faecium B2-9 | Seafood | − |
| E. faecium B2-15 | Seafood | − |
| E. faecium B2-18 | Seafood | − |
| E. faecium B2-21 | Seafood | − |
| E. faecium B2-23 | Seafood | − |
| E. faecium B2-25 | Seafood | − |
| E. faecium B2-31 | Seafood | − |
| E. faecium B2-37 | Seafood | − |
| Streptococcus canis 1001-3 | Clinical | − |
| S. dysgalactiae 814 | Milk | − |
| S. dysgalactiae 822 | Milk | − |
| S. equinus | Horse | − |
| S. equinus 866 | Horse | − |
| S. equisimilis 834-4 | Clinical | − |
| S. equisimilis 888-2 | Clinical | − |
| S. pneumoniae | Clinical | − |
| S. pyogenes ATCC 19615 | Human | − |
| S. pyogenes MGAS159 | Human | − |
| S. uberis 815 | Clinical | − |
| S. uberis 820 | Clinical | − |
| S. zooepidemicus 861-1 | Horse | − |
| S. zooepidemicus 865-2 | Horse | − |
| Staphylococcus aureus ATCC 25923 | Clinical | − |
| S. aureus 814 | Milk | − |
| S. cohnii 4850 | Chicken | − |
| S. epidermidis 979 | Human | − |
| S. epidermidis 986 | Human | − |
| S. haemolyticus 2168 | Human | − |
| S. hominus 1961 | Human | − |
| S. hominus 2130 | Human | − |
| S. Saccharolyticus 956 | Human | − |
| S. intermedius 840 | Dog | − |
| S. intermedius 845 | Dog | − |
| S. siuri 4856 | Squirrel brain | − |
| Aeromonas hydrophila ATCC 35654 | | − |
| Bordetella avium 2-1178 | Poulrtry | − |
| B. bronchiseptica | Pig | − |
| Clostridium perfringens | Clinical | − |
| Escherichia coli ATCC 25922 | Clinical | − |
| Flavobacterium indolegenes | Clinical | − |
| Klebsiella pneumoniae ATCC 13883 | | − |
| Listeria grayi ATCC 19120 | Corn leaves/stalks | − |
| L. grayi ATCC 25400 | Corn leaves/stalks | − |
| L. innocua ATCC 33090 | Cow brain | − |
| L. innocua ATCC 43547 | | − |
| L. ivanovii ATCC 19119 | Sheep | − |
| L. ivanovii SLCC 6965 | FDA | − |
| L. monocytogenes EGD (NCTC 7973) | Guinea pig | − |
| L. monocytogenes ATCC 19111 | Poultry | − |
| L. monocytogenes ATCC 19114 | Human | − |
| L. monocytogenes HCC 23 | Catfish | − |
| L. seeligeri ATCC 35967 | Soil | − |
| L. seeligeri SLCC 5921 | FDA | − |
| L. welshimeri ATCC 35897 | Plant | − |
| L. welshimeri ATCC 43550 | Soil | − |
| Mannheimia haemolytica D139 | Bovine lung | − |
| Micrococcus sedentarius 2134 | Human | − |
| Pasteurella multocida ATCC 11039 | Domestic fowl | − |
| Proteus vulgaris ATCC 13315 | | − |
| Pseudomonas aeruginosa ATCC 27853 | Blood culture | − |
| Salmonella typhimurium ATCC 14028 | | − |
| Serratia marcescens ATCC 8100 | | − |
| Yersinia pseudotuberculosis | Clinical | − |

### EXAMPLE 6

### PCR Detection of Pathgenic Leptospira Genomospecies Targeting Putative Transcriptional Regulator Genes

Bacterial Strains.

Leptospira and other reference bacterial strains were acquired from the USDA National Veterinary Services Laboratory (NVSL) or the American Type Culture Collection (ATCC). Twenty eight Leptospira strains representing seven pathogenic (ie, L. interrogans, L. alexanderi, L. borgpetersenii, L. kirschneri, L. kirschneri, L. noguchii, L. santarosai and L. weilii) and four non-pathogenic (ie, L. biflexa, L. inadai, L. meyeri and L. wolbachii) species were examined (Table 8). In addition, 46 other bacterial species/strains were included for assessment of the specificity of Leptospira primers developed (Table 9). Leptospira strains were cultured in EMJH broth supplemented with 10% rabbit serum (Difco Laboratories, Detroit, Mich.) and maintained by weekly subculture into fresh medium. For DNA isoaltion, Leptospira cultures were cultivated for seven days at 30° C. to stationary phase to a density of approximately 2×10⁸ cells per ml. Cells were harvested by centrifugation, resuspended in 0.15 M PBS pH 7.2 and stored at −20° C. prior to DNA extraction. Other bacteria were initially grown on 5% sheep blood agar plates (TSA II, Becton Dickinson Microbiology Systems, Cockeysville, Md.), and batch cultures in brain heart infusion (BHI) broth (Difco Laboratories, Detroit, Mich.) were maintained at 37° C. with rotary aeration. Genomic DNA.

Bacterial DNA was prepared using the method described in Example 1.

Identification of Leptospira Specific Gene(s).

The gene sequences of L. interrogans serovar icterohaemorrhagiae strain Lai that encode transcriptional regulators were retrieved from the published genome data (Ren, et al., Nature, 422:888-893 (2003)), and BLAST searches were conducted to identify transcriptional regulator genes that demonstrate no homology with other gene sequences at GenBank. As a result, eleven genes encoding putative transcriptional regulators or hypothetic proteins (ie, La0825, la0954, la1937, la2032, la2640, la2894, la3133, la3152, la3231, la3825 and la4130) were selected from the large circular chromosome (CI) of L. interrogans serovar Icterohaemorrhagiae strain Lai (Table 10). Interestingly, transcriptional regulator gene la1937 is essentially the same as la2032 and la3152 in L. interrogans serovar Icterohaemorrhagiae (Ren et al., Nature, 422:888-893, 2003)). Primers were designed from the nine genes by using Primer 3 software (Whitehead Institute for Medical Research, Cambridge, Mass.), and custom synthesized (Sigma Genosys, The Woodlands, Tex.) (Table 10).

PCR Amplification.

PCR was performed in a volume of 25 μl using a GeneAmp PCR System 9700 (Perkin Elmer, Foster City, Calif.). The reaction mixture was made up of 0.5 U Taq DNA polymerase (Fisher Scientific, Houston, Tex.), 1× PCR buffer (10 mM Tris-HCl pH 9.0, 50 mM KCl and 1.5 mM MgCl₂), 50 μM dNTPs, 25 pmol primers each and 15 ng DNA. A reaction mixture with no template DNA was used as a negative control in each run. The cycling programs consisted of 1 cycle of 94° C. for 2 min.; 30 cycles of 94° C. for 20 sec., 55° C. for 20 sec. and 72° C. for 45 sec.; and a final incubation at 72° C. for 2 min. After completion of all cycles, 3 μl of 10× DNA loading buffer was added to each tube, and the amplified products were examined in 1.0% agarose gel electrophoresis in the presence of ethidium bromide (0.5 μg/ml). The stained gels were visualized under UV light and results recorded using a Chemilmager 5500 (BSI, Stafford, Tex.).

Results.

Using PCR primers from the nine putative transcriptional regulator or hypothetic protein genes (ie, La0825, la0954, la1937, la2640, la2894, la3133, la3231, la3825 and la4130) resulted in the amplification of specific products from 19 of the 24 Leptospira pathogenic strains (with the exception of L. interrogans serovar Ballum S-102, L. interrogans serovar Mini Szwajizak, L. interrogans serovar Tarassovi Perepelicin, L. interrogans serovar Sejroe, and L. santarosai serovar Sherrnani) (Table 8; data not shown). None of the primers above reacted with Leptospira non-pathogenic strains (ie, L. biflexa, L. inadai, L. meyeri and L. wolbachii), the other 44 common bacterial species/strains, or the negative (no DNA template) control in PCR (Table 8; data not shown).

These results show that the nine putative transcriptional regulator and hypothetic protein genes (ie, La0825, la0954, la1937, la2640, la2894, la3133, la3231, la3825 and la4130) are only present in pathogenic Leptospira species/strains, and hence can be used as markers for the detection of these bacteria strains. From a previous study with foodborne pathogen L. monocytogenes, it was noted that transcriptional regulator genes have some important roles to play in listerial virulence, since L. monocytogenes avirulent strains possess fewer such genes that virulent strains (Liu et al., J. Med. Microbiol., 52:1065-1070 (2003)). Therefore, similar to nonpathogenic Leptospira species, the five Leptosipra strains (ie, L. interrogans serovar Ballum S-102, L. interrogans serovar Mini Szwajizak, L. interrogans serovar Tarassovi Perepelicin, L. interrogans serovar Sejroe, and L. santarosai serovar Shermani) without the nine transcriptional regulator and hypothetic protein genes (ie, La0825, la0954, la1937, la2640, la2894, la3133, la3231, la3825 and la4130) may be much less virulent than the others containing these genes.

TABLE 8
Examination of Leptospira species/strains by PCR targeting pathogen-specific genes

| | | PCR targeting La0825, la0954, |
|---|---|---|
| | | la1937, la2640, la2894, la3133, |
| Species/strains | Source | la3231, la3825, la4130 |
| L. interrogans serovar Australis, | NVSL ARL-10 | + |
| Ballico | | |
| L. interrogans serovar Autumnalis, | NVSL ATL-10 | + |
| Akiyami A | | |
| L. interrogans serovar Ballum, S-102 | NVSL BML-11 | − |
| L. interrogans serovar Batavie, van | NVSL BTL-020 | + |
| Tienen | | |
| L. interrogans serovar Bratislava, Jez | NVSL ARL- | + |
| Bratislava | 050 | |
| L. interrogans serovar Canicola, Hond | NVSL CAL- | + |
| Utrecht IV | 010 | |
| L. interrogans serovar Grippotyphosa, | NVSL GRL- | + |
| Andaman | 020 | |
| L. interrogans serovar Hardjo, Hardjio | NVSL SJL-060 | + |
| prajtino | | |
| L. interrogans serovar Hebdomadis, | NVSL HBL- | + |
| Hebdomadiz | 010 | |
| L. interrogans serovar Copenhageni, | NVSL ICL-020 | + |
| MZO | | |
| L. interrogans serovar Mini, Szwajizak | NVSL MIL-020 | − |
| L. interrogans serovar Pomona, Pomona | NVSL POL-010 | + |
| L. interrogans serovar Pyrogenes, | NVSL PYL-010 | + |
| Salinem | | |
| L. interrogans serovar Tarassovi, | NVSL TAL-10 | − |
| Perepelicin | | |
| L. interrogans serovar Sejroe, M84 | NVSL SJL-010 | − |
| L. alexanderi serovar Manhao 3 | ATCC 700520 | + |
| L. borgpetersenii serovar Javanica | ATCC 23479 | + |
| L. kirschneri serovar Grippotyphosa | ATCC 23469 | + |
| L. kirschneri | ATCC 23579 | + |
| L. kirschneri serovar Cynopteri | ATCC 49945 | + |
| L. noguchii serovar Panama | ATCC 43288 | + |
| L. santarosai serovar Borincana | ATCC 23477 | + |
| L. santarosai serovar Shermani | ATCC 43286 | − |
| L. weilii serovar Celledoni | ATCC 43285 | + |
| L. biflexa serovar Patoc | ATCC 23582 | − |
| L. inadai serovar Lyme | ATCC 43289 | − |
| L. meyeri serovar Ranarum | ATCC 43287 | − |
| L. wolbachii serovar Codice | ATCC 43284 | − |

TABLE 9
List of common bacterial species used in the present invention

| Strain | Source |
|---|---|
| Aeromonas hydrophila ATCC | |
| 35654 | |
| Bordetella avium 2-1178 | Poultry |
| Bordetella bronchiseptica | Pig |
| Clostridium perfringens | Clinical |
| Escherichia coli ATCC | Clinical |
| 25922 | |
| Enterococcus faecalis ATCC | |
| 29212 | |
| Enterococcus faecium ATCC | |
| 349 | |
| Enterococcus avium ATCC | |
| 14025 | |
| Enterococcus casseliflavus | |
| ATCC 25788 | |
| Enterococcus durans ATCC | |
| 19432 | |
| Enterococcus gallinarum | |
| ATCC 49573 | |
| Enterococcus hirae ATCC | |
| 8043 | |
| Enterococcus mundtii ATCC | |
| 43186 | |
| Enterococcus raffinosus | |
| ATCC 49427 | |
| Flavobacterium indologenes | Clinical |
| Klebsiella pneumoniae ATCC | |
| 13883 | |
| Listeria grayi ATCC 19120 | Corn |
| Listeria innocua ATCC | Cow brain |
| 33090 | |
| Listeria ivanovii ATCC | Sheep |
| 19119 | |
| Listeria monocytogenes EGD | Guinea pig |
| (NCTC 7973) | |
| Listeria seeligeri ATCC | Soil |
| 35967 | |
| Listeria welshimeri ATCC | Plant |
| 35897 | |
| Mannheimia haemolytica | Bovine |
| D139 | lung |
| Micrococcus sedentarius 2134 | Human |
| Pasteurella multocida ATCC 11039 | Domestic |
| | fowl |
| Proteus vulgaris ATCC 13315 | |
| Pseudomonas aeruginosa ATCC | Blood culture |
| 27853 | |
| Salmonella typhimurium ATCC | |
| 14028 | |
| Serratia marcescens ATCC 8100 | |
| Staphylococcus epidermidis ATCC | |
| 12228 | |
| Staphylococcus aureus ATCC 25923 | |
| Staphylococcus cohnii 4850 | Chicken |
| Staphylococcus haemolyticus 2168 | Human |
| Staphylococcus hominus 1961 | Human |
| Staphylococcus saccharolyticus 956 | Human |
| Staphylococcus intermedius 840 | Dog |
| Staphylococcus siuri 4856 | Squirrel brain |
| Streptococcus canis 1001-3 | Clinical |
| Streptococcus dysgalactiae 814 | Milk |
| Streptococcus equinus | Horse |
| Streptococcus equisimilis 834-4 | Clinical |
| Streptococcus pneumoniae | Clinical |
| Streptococcus pyogenes ATCC 19615 | |
| Streptococcus uberis 815 | Clinical |
| Streptococcus zooepidemicus 861-1 | Clinical |
| Yersinia pseudotuberculosis | Clinical |

TABLE 10
Identities of Leptospira putative transcriptional regulator and hypothetic protein
genes and primers

| | | Nucleotide | | | |
|---|---|---|---|---|---|
| | | positions | | | |
| | | (Genbank | | | PCR |
| | Primer | Accession No. | Putative | Primer sequence | product |
| Gene | position | AE010300) | protein function | (5′-3′) | (bp) |
| La4130 | 4961-4980; | 4097575-4098102 | Transcriptional | aagttttaaccttcgcatttga | 410 |
| | | | regulator | (SEQ ID NO: 15) | |
| | 5533-5514 | | | cgcaatgaattccaatttacc | |
| | | | | (SEQ ID NO: 16) | |
| La0825 | 8104-8135; | 826837-827451 | Transcriptional | ttcacgaagagaatcgtcca | 414 |
| | | | regulator | (SEQ ID NO: 17) | |
| | 8513-8493 | | | aaagagctgggacttgcgta | |
| | | | | (SEQ ID NO: 18) | |
| La0954 | | 948414-949739 | Hypothetical | ttgcagttggtggtaacgaa | 557 |
| | | | protein | (SEQ ID NO: 19) | |
| | | | | cagagttgcatccggaattt | |
| | | | | (SEQ ID NO: 20) | |
| La1937* | 816-835; | 191032-1910877 | Transcriptional | gaccagtgaaacgtgggaac | 435 |
| | | | regulator | (SEQ ID NO: 21) | |
| | 1250-1231 | | | cgtagtgggtatgggcattt | |
| | | | | (SEQ ID NO: 22) | |
| La2032* | | 2008383-2008940 | Transcriptional | gaccagtgaaacgtgggaac | 435 |
| | | | regulator | (SEQ ID NO: 23) | |
| | | | | cgtagtgggtatgggcattt | |
| | | | | (SEQ ID NO: 24) | |
| La2640 | | 2624163-2624789 | Hypothetical | tctgaaatggccggattcta | 372 |
| | | | protein | (SEQ ID NO: 25) | |
| | | | | tcgcttttccaattgattttg | |
| | | | | (SEQ ID NO: 26) | |
| La2894 | | 2870942-2871553 | Transcriptional | gaatctgcgacttcgattcc | 420 |
| | | | regulator | (SEQ ID NO: 27) | |
| | | | | gcaaatgaacccagctatgg | |
| | | | | (SEQ ID NO: 28) | |
| La3133 | | 3117584-3118765 | Transcriptional | aattctccgctgggattttt | 698 |
| | | | regulator | (SEQ ID NO: 29) | |
| | | | | ttggttctggagttccaagg | |
| | | | | (SEQ ID NO: 30) | |
| La3152* | | 3136320-3136877 | Transcriptional | gaccagtgaaacgtgggaac | 435 |
| | | | regulator | (SEQ ID NO: 31) | |
| | | | | cgtagtgggtatgggcattt | |
| | | | | (SEQ ID NO: 32) | |
| La3231 | 4590-4611; | 3202437-3202841 | Transcriptional | caggatatattttgagcgagca | 409 |
| | | | regulator | (SEQ ID NO: 33) | |
| | 4977-4998 | | | ttttcgatttcttgtttttcca | |
| | | | | (SEQ ID NO: 34) | |
| La3825 | 4950-4969; | 3786562-3787506 | Transcriptional | cgcctttcgttcatgacaat | 490 |
| | | | regulator | (SEQ ID NO: 35) | |
| | 5420-5439 | | | cgaagaacttgccagtgtga | |
| | | | | (SEQ ID NO: 36) | |

The nucleotide sequence of putative transcriptional regulator gene la1937 is nearly identical to those of la2032 and la3152; and thus primers from la1937 will also recognize la2032 and la3152.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any squence which is effective to meet the objectives there intended, unless the context specifically indicates the contrary.

SEQ ID NOs 37-47 are nucleotide sequences of Leptospira pathogen-
specific genes (from Genbank Accession No. AE010300)
La0825 (826837-827451) (SEQ ID NO: 37)
826801 ataaaacttt aaaaggaaag tttttacgaa atttatttaa aggattctat attcttctac
826861 aatttcacga agagaatcgt ccatctcacg aataaaacga agttcttgag tcattctcca
826921 cattgtaatt gaaccgtggt atgccattaa aattcttctc gccacgtatt ggatgtccat
826981 atttcttttg agttgtcccg atccgatcgc cttttgcaga taatcgctga tcatacgaat
827041 ccatttgttt acgatgtctt ttaaaaattc agtatattca ggatcggagt ccattacttg
827101 acttgcaaaa ccagcaaaag gacaacctac aaattctccg tgacggattt gtttttcttt
827161 gagaatcacc caggctctta aaaattctcc taggtctgga taacgatcca tcaaactttg
827221 aagatcgcta agagtttttt cttcctgacg agctagatac gcaagtccca gctctttttt
827281 agaagggtag tgatcgtaga gacttgcagc aacagaattg gattcctgaa tgatttgcct
827341 catacctgta ctggaaaaac cttgtttgta aaaaagagaa gtagcggtat ctaaaattct
827401 ttcccgaact ccagttccgg attctttttt tttacgcggt tgagttttca tagaggaacg
827461
La0954 (948414-949739) (SEQ ID NO: 38)
948361 ccaatcaaag cgtaagttcg tcttgttgaa cttaacaaac gcaaattgat cctatgtccg
948421 gaaatgataa aaagattcta gaacaaaatt ctcacgggga atttgaacta acagcttacg
948481 gagaattctt aagttatttc catactcata ttcaactttt taacggtctt atctctggta
948541 agaaaatttc tcccacagac caggaagttc ttaaacaaaa agtacgttct tatattgtaa
948601 gtaatatcca aaagacggaa caattttttg atcacctgcc taagttcgca gaatttctag
948661 gaatgtccca atctgatctt tccgcgttta tgacgaaaaa cttcatgaac acgcacgctg
948721 ggatcaaaaa caaactcatc gaacaggaaa aggcaaacat aggtaaacct aaaaagaaaa
948781 gatattcccg catttcggaa gaaattttgg aaacgatcgg agaattggtt cctccaggta
948841 aaaggtttat cggaatggaa ggttatgtag tccttaggga cgatgcgact ggaaaagatc
948901 ttgaaccttc tatttcttct ttcggcgaaa cacctaaaca aacgaacgag gaaaatacgc
948961 cgggtgtcgt taaaaaaaca gcaccacaag cttctacccc cgccgtgcct ttgaaaaagc
949021 agactgagaa attgattctt actgagatct tggaaaaatt tggttctgaa ttttccggaa
949081 aggttttaga attaaaaaag gaagagtttg aggaagcgga aataaccaat caaattgcag
949141 ttggtggtaa cgaagaattg ttaaccgaag tagaggatct tcaatttgaa ggtttcgaag
949201 agcctacttt tttagaagat acttcgtttg aaccggaaga gccacctgtg aatattccgt
949261 tttctaaata tatggaaagt ataaaccgtg ttcgacagtt tcaaaaagac ggtcaaccgg
949321 acgcatataa aaaatgggtg atggctcttc ctccggaact cagtgcactt gttcaacttc
949381 attcttttgt tttaaaggaa atgaaaaacg aacctttgga ttggaattct accctttcct
949441 cgatttcttc ccgaatggga ttaacggaga aaagactttg gaaggtttta gatttgaccc
949501 gtatttttgc cgaactcagg gcaggtttgg agaaggcttt tgtatcttct aaaactgctg
949561 gtccaggaat ggaagaactt gtaaaaaaag cctggcctca tattctcaaa atcttcgaag
949621 aatacccaaa tacttcttct ttgcaccaaa agttagatca actttttagt aaaattccgg
949681 atgcaactct gagaaaaaaa ctttccgatc tatttcttcc tcttcttcaa aaactctgat
949741
La1937 (191032-1910877) (SEQ ID NO: 39)
1910281 atgagtttta aatttgatga tagccgccta atagtgattt tatgtagaaa accactctaa
1910341 gtctaatgcg taaaaatagg attctggttc acagtgtagt tttcgagtga cttggttgtt
1910401 tgacagatct aggtgcagga tataactgta ggaccagtga aacgtgggaa ctcccgcccg
1910461 aaccgtattc aagatagaat taccgactcc gagggcttcc aactttaaaa gataattaaa
1910521 aagataacat tttgatacac cgtgagcctg agccaatgtt cccaaaagag tccaacttgc
1910581 agaaggaaca cgaacgctga ttcgtttcat ttttaatttg cctggactgg gttgatacaa
1910641 agttctgtcc gcttttttac cgagacgttc cgacgaagaa agatatttcg cataaacttt
1910701 caaaagtgta gggatccttt tcgtaagtag cctcacgtct ttttcagaaa aaagaagcca
1910761 agtatcttcc ggaattaaaa gagtcaccgt ttcagaacaa ttcttttgga atgtagaact
1910821 aatttcatga tcagagttga gtaataaaat gcccataccc actacggttc tcgacaagag
1910881
La2032 (2008383-2008940) (SEQ ID NO: 40)
2008381 ttttatgtag aaaaccactc taagtctaat gcgtaaaaat aggattctgg ttcacagtgt
2008441 agttttcgag tgacttggtt gtttgacaga tctaggtgca ggatataact gtaggaccag
2008501 tgaaacgtgg gaactcccgc ccgaaccgta ttcaagatag aattaccgac tccgagggct
2008561 tccaacttta aaagataatt aaaaagataa catttcgata caccgtgagc ctgagccaat
2008621 gttcccaaaa gagtccaact tgcagaagga acacgaacgc tgattcgttt catttttaat
2008681 ttacccggac tatgttgata caaagttctg tccgcttttt taccgagacg tttcgacgaa
2008741 gaaagatatt tcgcataaac cttcaaaagt gtagggatcc ttttcgtaag tagcctcacg
2008801 tctttttcag aaaaaagaag ccaagtatct tccggaatta aaagagtcac cgtttcagaa
2008861 cgattctttt ggaatgtaga actaatttca tgatcagagt tgagtaataa aatgcccata
2008921 cccactacgg ttctcgacaa gagtaagacg gcttaaattt gaatcaaaaa aatcactcat
2008981
La2640 (2624163-2624789) (SEQ ID NO: 41)
2624161 atttgaaacc taaaatagca attcaaatct acaaaagtag actcatttta tcaaaagaag
2624221 accgtggttt tcaaaataga tccgttctga aatggccgga ttctattgaa tcttctacat
2624281 ctaaaataga agacgattgt gtccgcttta tttccaatct agattttagt acaacagaat
2624341 cgtttgcaaa ttcagaattt ttcgttactt tagattatca tccagagact aaaattccca
2624401 aaaaccagtt ctatgacgat cttgacgaag tttcatttaa cttttcaaaa ttaactagat
2624461 tcaaatttga tatcttcgaa tttgaaaaaa tgagttcgga tcaagtaaat ttatttttta
2624521 atggaacaaa tttgggtatt cctaaaagac aatcttttca aatagccagc ctaagtctaa
2624581 attcaccggt tagaatcaaa atcaattgga aaagcgactt ttctttttct cagggaaaag
2624641 aaagacaatt ttacgaacta gattatatcg ttgaatttaa aggtatattt gaaacttttt
2624701 tgagagaaaa tcccaaaaca gaaacctttc tcaaatatat ccaaatatcg aactgtaaag
2624761 aaatagatct tagaaaaatt ctcaattaat acgtgtttga tgcaaaaaaa ttagattttt
2624821
La2894 (2870942-2871553) (SEQ ID NO: 42)
2870941 tttaattgat ttcatcccta taatccataa ccaaatacat tagagtttct tgatctgttg
2871001 gattggaata ttcatgagga atatcggctc tgaaaaaaac ggaatctcta ggttccaact
2871061 ctacaacttt ttcaccaacc ctaagacgaa gtttaccgga aacaacaaca atgttttcag
2871121 tagtgcctga tgagtgggaa tctgcgactt cgattcctcc cggttttaaa atgagttcat
2871181 aaaactctgt ttttcgattt ccgttataag gaaaaagagc tcggctggaa aaaactttag
2871241 agctagaaaa caaaacctta gtgttttccg ccttcataac aattacacct tcggtacctt
2871301 tttctttgag taattcgcta aaaggcacat tgagtccatt cgcaattttc caaagaacgg
2871361 aaatggtcgg aacacttttc ccttgttcaa tttgagaaag catcgccctg ctgactccac
2871421 atctagaagc cagtttgtcc aaagagaacc ctttagtatg gcgaatgagt tttaaatttt
2871481 ctttgacgac ttcagtaata tgttcggagg aagactcttt cccctcttct aattcttcca
2871541 tagctgggtt catttgctct tttttatcta atatagcaga aatttgtcaa tccaaaacta
2871601
La3133 (3117584-3118765) (SEQ ID NO: 43)
3117541 caagttatct taggtcaaaa gaattgttag aggtgagagt agaatggatt tttttccgga
3117601 tctgaatttt cttgattatt taatttttgt aaaagaagta ttaaaccggt atctagaata
3117661 tctttactct gcaagtatag taattagttt tttaaccggt ctttctggtt tttacaaagc
3117721 aaccaaaaat cgtttaaact ttgttcgagg aattttttta atgagcgctt gttttctaat
3117781 tatcggacac agtaaagctt tttcattgat gacttcggat ccaaatggaa ttttaagtct
3117841 cgaaaagcgt gtcttttttt cetattacta cggatttgca gtttgtgcct ctttatcttc
3117901 caccttctac gtcatgtatc tttttggtgc tttgaaaaat cctctacgtt tgagcctata
3117961 tagttttctt tcttttccaa caatctttct tatttcttta atgatagaaa acttaatttc
3118021 aatcgtttac tttggaaagt cagttctttt taccttacaa ttctccgctg ggatttttaa
3118081 tctttattat gttatcaaat ataagatgaa aaaaatctat ctcaattatc caattcaaaa
3118141 ttttacactt tgtacagcgc ttatcgtaca ttcgatcgga atgtggttag gcactccaaa
3118201 cttgatgatt cttgcacttt cttttacagg atttttcatt atttattttt tcatattgga
3118261 atacaatcat cctgaatttt ggaaagtgag tttttccagc gaactgatag aaattaaatc
3118321 agaagataat aaaattcaaa ctctacaaac caatactagt tctaaaaatt tagtagaacg
3118381 tttagacata tctcgtatcg aagaaaaaat tcaaaaattt gtggaagata gagaatattt
3118441 agacgaagaa attcgcttgt ccgatttttc agcttatatc ggactttcac ttcaccaagc
3118501 ttcttattat ttgaacaatt ataaggatct cagctttaca gatttcttaa gcttccatag
3118561 actagaagaa gcaaaacgta tgattgaaca aagaccagat attaatcttt tagaagtggc
3118621 tttagcaagc ggtttcaatt ctccttcttc ttttagaaga gcttgtctaa aatttaccgg
3118681 aaaacctcca aaagaattta gaaactacgt acttcatcaa acaactcctt ctattacctt
3118741 ggaactccag aaccaattag gataattttt tcgttccatc taatttgatt gatagaacga
3118801
La3152 (3136320-3136877) (SEQ ID NO: 44)
3136261 aatctaggaa cttctacgaa atgagtttta attttgatga taaccgccta atagtgattt
3136321 tatgtagaaa accactctaa gtctaatgcg taaaaatagg attctggttc acaatgtagt
3136381 tttcgagtga cttggttgtt tgacagatct atgtccagga tataactgta agaccagtga
3136441 aacgtgggaa ctcccgcccg aaccgtattc aagatagaat taccgactcc gagggcttcc
3136501 aactttaaaa gataattaaa aagataacat ttcgatacac cgtgagcctg agccaatgtt
3136561 cccaaaagag tccaacttgc agaaggaaca cgaacgctga ttcgtttcat ttttaattta
3136621 cctggactgg gttgatacaa agttctgtcc gcttttttac cgagacgttc cgacgaagaa
3136681 agatatttcg cataaacttt caaaagtgta gggatccttt tcgtaagtag cctcacgtct
3136741 ttttcagaaa aaagaagcca agtatctttc ggaattaaaa gagtcaccgt ttcagaacga
3136801 tttttttgaa gtgcggaact aatttcatga tcagaattga gtaataaaat gcccataccc
3136861 actacggttc tcgacaagag taagacggct caaatttgaa tcaaaaaaat cactcattca
3136921
La3231 (3202437-3202841) (SEQ ID NO: 45)
3202381 gattagtatt attttaatac taatgtcaat taaaaagtat tattccagga tatattttga
3202441 gcgagcaaaa aaaaagacta aaaaccatac ttttagattt caaaggaaat caaagggagt
3202501 ttggagtcac aattggcaaa tccaaacaga caatcagtgg ctggctaagt ggtagatttc
3202561 caataccaga ggatgctgca attacaatag aaatggttca tggataccga agacaatggc
3202621 ttctcgaagg agaacttcca gaaaaagtaa ctagacgaat acaaacatct cgaacaagaa
3202681 caaaagaatt tgaactagaa aagactttgt taaaaaaaat aacttctaag gagggccttc
3202741 ctaaaatgat tgaaatactt acagtacttc caaaaaaaga atttgaaatc gcccaaagat
3202801 ttatttttag cctggaaaaa caagaaatcg aaaacaatta gaatatatcc tatcaagaat
3202861
La3825 (3786562-3787506) (SEQ ID NO: 46)
3786541 aaaacattgg cacgatcgta tctaatgaag aatcgattct aattctctca gaatccgctt
3786601 tgccaaatga gtaggacgtt ccaaaattac attttttcca aatggaagaa ttgtttccaa
3786661 aaaccagttt tcttcccgaa ttttggcttt ggataaatga taaacgacat tatccaattt
3786721 ttgagttttt tcggttcttt ccaaatcaag tttacgattg agattataaa acacttcccg
3786781 agtatgccaa atttctgcga ttccggatga attctcccaa cttttacgaa attcttcaaa
3786841 ttctcgaata taatctgatt ttttttgacc agctggttgc aagatcgggt ccgaaccaat
3786901 ttttaaggaa agaatattgt ccaagcgaaa atttcgaggc gcctttcgtt catgacaata
3786961 tcctaataaa taatcttcta atgaatgaaa taaaaaccaa ggatctactt ttctaagagt
3787021 agatttttcg cccattctgg attgatattc caggattaaa gatttttcgt tttgaatcgc
3787081 attttgaata ttagttttgt aaaccgaaag tgcttcctgg ccagcaatgg gaacaataga
3787141 aatgattttc tgaagtattt tacgagcagt tgaatttgtt tttggatcgg agctggcttc
3787201 tacaacttct tctaaaattt ttctaagtgt tagccattct cgaatactca aacgtaaact
3787261 agaatcaaaa cgaaaaggta aaatcaaacc aaatgtatcc gtttctgaat cataatcgac
3787321 ttggatcaaa tcggcgacgt gaggagttgt ccccagaaaa aaaagttctc ccaattgttc
3787381 ttttaaatct ttttgattgt tgattccggt cacactggca agttcttcga gagtcattcg
3787441 tttaccatct cgaagatggc gaatcagatt caatttaaaa ttcagtcgaa ctgtacttgg
3787501 attcacacct caaatgtaaa aatggcacac attctgtaaa ccggaaatgt aagaatcttg
La4130 (4097575-4098102) (SEQ ID NO: 47)
4097521 attttaggct ctattcaaca attgtatgaa taaaattttc tttagaaggt aaaaatgaaa
4097581 ttttggtttc cacaagaaag acgattttat tttttttcga tgtatatctt tttaatttta
4097641 ctttggatca ttgaagaagt tttaaccttc gcatttgata ttaattggat agaaagatct
4097701 caggcttatt ttactacgat cgaagcagca ttcggtctgt tatctattat aggaatttat
4097761 tttttatttc aagaaattag aaacacaaaa acggatattg aatctgctaa aataatgatc
4097821 gaaggactca aaaataaaaa tcaattttta gttcaaacca atcaatcttt ctgggaatct
4097881 cttcaaagac aattggaaga atgggacctc tccgataaag aaaaagaaat tgcactttta
4097941 cttctaaggg gtatgtctaa tcaccaaatt gctgctattc gaggaaaaag tttaaaaaca
4098001 atcgagaacc aaacattttc tatctatcaa aaatctggaa ctactggtaa attggaattc
4098061 attgcgtatt tcatttctcc tttactaccc gaagaagatt gatctataat ctttctcgta
4098121

1. A method for detecting the presence of bacteria in a sample, said method comprising:
subjecting said sample to polymerase chain reaction (PCR) amplification using primers designed to target a transcriptional regulator gene or a putative transcriptional regulator gene specific for a bacterial strain; and
detecting the presence of an amplification product of said transcriptional regulator gene or putative transcriptional regulator gene as an indication of the presence of said bacterial strain;
wherein said bacterial strain is selected from the group consisting of Pasteurella multocida, Staphylococcus aureus, Streptococcus pyogenes, Enterococcusfaecalis, and Leptospira strains.

2. The method of claim 1, wherein said bacterial strain is Pasteurella multocida and said putative transcriptional regulator gene is one of Pm0762 and Pm1135.

3. The method of claim 2, wherein said putative transcriptional regulator gene is Pm0762, and wherein said primers comprise a first primer comprising the oligonucleotides sequence recited in SEQ ID NO:3 and a second primer comprising the oligonucleotides sequence recited in SEQ ID NO:4.

4. The method of claim 2, wherein said putative transcriptional regulator gene is Pm1135, and wherein said primers comprise a first primer comprising the oligonucleotides sequence recited in SEQ ID NO:5 and a second primer comprising the oligonucleotides sequence recited in SEQ ID NO:6.

5. The method of claim 1, wherein said bacterial strain is Staphylococcus aureus and the putative transcriptional regulator gene is Sa0836 or Sa0856.

6. The method of claim 5, wherein said putative transcriptional regulator gene is Sa0836, and wherein said primers comprise a first primer comprising the oligonucleotides sequence recited in SEQ ID NO:7 and a second primer comprising the oligonucleotides sequence recited in SEQ ID NO:8.

7. The method of claim 5, wherein said putative transcriptional regulator gene is Sa0856, and wherein said primers comprise a first primer comprising the oligonucleotides sequence recited in SEQ ID NO:9 and a second primer comprising the oligonucleotides sequence recited in SEQ ID NO:10.

8. The method of claim 1, wherein said bacterial strain is Streptococcus pyogenes and the putative transcriptional regulator gene is Spy1258

9. The method of claim 8, wherein said primers comprise a first primer comprising the oligonucleotides sequence recited in SEQ ID NO:11 and a second primer comprising the oligonucleotides sequence recited in SEQ ID NO:12.

10. The method of claim 1, wherein said bacterial strain is Enterococcus faecalis and said putative transcriptional regulator gene is Ef0027

11. The method of claim 10, wherein said primers comprise a first primer comprising the oligonucleotides sequence recited in SEQ ID NO:13 and a second primer comprising the oligonucleotides sequence recited in SEQ ID NO:14.

12. The method of claim 1, wherein said bacterial strain is a pathogenic Leptospira strain and said putative transcriptional regulator or hypothetic protein gene is selected from the group consisting of la0825, la0954, la1937, la2032, la2640, la2894, la3133, la3152, la3231, la3825, and la4130.

13. The method of claim 12, wherein said putative transcriptional regulator gene is la4130, and wherein said primers comprise a first primer comprising the oligonucleotides sequence recited in SEQ ID NO:15 and a second primer comprising the oligonucleotides sequence recited in SEQ ID NO:16.

14. The method of claim 12, wherein said putative transcriptional regulator gene is la0825, and wherein said primers comprise a first primer comprising the oligonucleotides sequence recited in SEQ ID NO:17 and a second primer comprising the oligonucleotides sequence recited in SEQ ID NO:18.

15. The method of claim 12, wherein said putative transcriptional regulator gene is la0954, and wherein said primers comprise a first primer comprising the oligonucleotides sequence recited in SEQ ID NO:19 and a second primer comprising the oligonucleotides sequence recited in SEQ ID NO:20.

16. The method of claim 12, wherein said putative transcriptional regulator gene is la1937, and wherein said primers comprise a first primer comprising the oligonucleotides sequence recited in SEQ ID NO:21 and a second primer comprising the oligonucleotides sequence recited in SEQ ID NO:22.

17. The method of claim 12, wherein said putative transcriptional regulator gene is la2032, and wherein said primers comprise a first primer comprising the oligonucleotides sequence recited in SEQ ID NO:23 and a second primer comprising the oligonucleotides sequence recited in SEQ ID NO:24.

18. The method of claim 12, wherein said putative transcriptional regulator gene is la2640, and wherein said primers comprise a first primer comprising the oligonucleotides sequence recited in SEQ ID NO:25 and a second primer comprising the oligonucleotides sequence recited in SEQ ID NO:26.

19. The method of claim 12, wherein said putative transcriptional regulator gene is la2894, and wherein said primers comprise a first primer comprising the oligonucleotides sequence recited in SEQ ID NO:27 and a second primer comprising the oligonucleotides sequence recited in SEQ ID NO:28.

20. The method of claim 12, wherein said putative transcriptional regulator gene is la3133, and wherein said primers comprise a first primer comprising the oligonucleotides sequence recited in SEQ ID NO:29 and a second primer comprising the oligonucleotides sequence recited in SEQ ID NO:30.

21. The method of claim 12, wherein said putative transcriptional regulator gene is la3152, and wherein said primers comprise a first primer comprising the oligonucleotides sequence recited in SEQ ID NO:31 and a second primer comprising the oligonucleotides sequence recited in SEQ ID NO:32.

22. The method of claim 12, wherein said putative transcriptional regulator gene is la3231, and wherein said primers comprise a first primer comprising the oligonucleotides sequence recited in SEQ ID NO:33 and a second primer comprising the oligonucleotides sequence recited in SEQ ID NO:34.

23. The method of claim 12, wherein said putative transcriptional regulator gene is la3825, and wherein said primers comprise a first primer comprising the oligonucleotides sequence recited in SEQ ID NO:35 and a second primer comprising the oligonucleotides sequence recited in SEQ ID NO:36.
